(19) United States
(12) Patent Application Publication
(10) Pub. No.: US 2022/0356476 A1
(43) Pub. Date: Nov. 10, 2022

(54) COMPOSITIONS AND METHODS USEFUL FOR EBOLA VIRUS INFECTION

(71) Applicant: AIM ImmunoTech Inc., Ocala, FL (US)

(72) Inventors: David R. STRAYER, Ocala, FL (US); Thomas K. EQUELS, Ocala, FL (US)

(73) Assignee: AIM ImmunoTech Inc., Ocala, FL (US)

(21) Appl. No.: 17/621,648

(22) PCT Filed: Jul. 2, 2020
(86) PCT No.: PCT/US2020/040655

§ 371 Date: Dec. 21, 2021

Related U.S. Application Data
(60) Provisional application No. 62/870,377, filed on Jul. 3, 2019.
(60) Provisional application No. 62/870,384, filed on Jul. 3, 2019.

(51) Int. Cl. C12N 15/117; A61K 38/21; A61K 31/713; A61K 39/12; A61P 31/14

(52) U.S. Cl. CPC C12N 15/117; A61K 38/212; A61K 31/713; A61K 39/12; A61P 31/14; C12N 2760/14123; C12N 2760/14134; C12N 2760/14162; C12N 2310/17; C12N 2320/31; A61K 2039/543

(57) ABSTRACT

Disclosed are methods and compositions for at least preventing, treating, inhibiting, or attenuating an Ebola virus infection of a subject. The methods comprise administering an effective amount of a composition as described herein to the subject thereby at least preventing, treating, inhibiting, or attenuating the Ebola virus infection of the subject. The compositions comprise a therapeutic double-stranded RNA (tdsRNA) and additional optional components such as an Ebola antigen.

### PRIORITY

This application claims the benefit of priority to U.S. Provisional Application 62/870,377 filed Jul. 3, 2019, and U.S. Provisional Application 62/870,384 filed Jul. 3, 2019, each of which is incorporated by reference herein.

### BACKGROUND

Infection by Ebola virus leads to Ebola Hemorrhagic Fever (EHF), the clinical manifestations of which are severe. An Ebola virus infection has an incubation period of four to sixteen days. The initial symptoms are generally a severe frontal and temporal headache, generalized aches and pains, malaise, and fever. Later and more severe symptoms include watery diarrhea, abdominal pain, nausea, vomiting, a dry and sore throat, and anorexia. By day seven after onset of the symptoms, the patient will often have a maculopapular (small, slightly raised spots) rash. At the same time, the person may develop thrombocytopenia and hemorrhagic manifestations, particularly in the gastrointestinal tract, and the lungs, but it can occur from any orifice, mucous membrane or skin site. Ebola virus infection causes lesions in almost every organ, although the liver and spleen are the most noticeably affected. Both are darkened and enlarged with signs of necrosis. The cause of death is normally shock, associated with fluid and blood loss into the tissues.

Susceptible hosts of Ebola virus include humans, non-human primates (monkey, gorilla and chimpanzee) and guinea pigs (which is a universally accepted model animal for study of the disease). The virus is transmitted to people from wild animals (possible natural hosts such as fruit bats, etc.) and spreads in the human population through human-to-human transmission. These human-to-human transmissions include direct contact (through broken skin or mucous membranes) with the blood, secretions, organs or other body fluids of infected people, and indirect contact with the environment contaminated with these fluids.

Because of the serious health issues associated with Ebola virus infection, there is an urgent need in this field for developing a drug capable of effectively inhibiting the transmission of Ebola virus. There are benefits for even short-term protection to allow protection of patients, doctors and laboratory workers who have to work with the virus or infected hosts.

### SUMMARY

One embodiment is directed to a method of at least preventing, treating, inhibiting, or attenuating an Ebola virus infection of a subject, the method comprising the step of: administering an effective amount of a composition comprising a tdsRNA; and a pharmaceutically acceptable carrier; to the subject thereby at least preventing, treating, inhibiting, or attenuating the Ebola virus infection of the subject.

The composition may be administered within a period of time from 96 hours before to 96 hours after exposure to Ebola virus; from 72 hours before to 72 hours after exposure to Ebola virus; from 48 hours before to 48 hours after exposure to Ebola virus; from 24 hours before to 24 hours after exposure to Ebola virus; from 12 hours before to 12 hours after exposure to Ebola virus; from 6 hours before to 6 hours after exposure to Ebola virus; from 3 hours before to 3 hours after exposure to Ebola virus; or from 1 hour before to 1 hour after exposure to Ebola virus. That is, administering is within the described period of time even though the administering itself may be a short time such as 30 seconds, one minute, five minutes, or 15 minutes.

Another embodiment is directed to a method of at least inhibiting, reducing or attenuating the replication of Ebola virus in a subject that was exposed to Ebola virus comprising the step of administering a composition comprising a tdsRNA; and a pharmaceutically acceptable carrier; to a subject within a period of time after the subject has been exposed to Ebola virus. The period of time may be selected from the group consisting of: 4 days, 3 days, 2 days, 1 day, 12 hours, 6 hours, 3 hours, and 1 hour.

Another embodiment is directed to the use of tdsRNA in an effective amount in the manufacture of a medicament for a subject for at least preventing, treating, inhibiting, or attenuating an Ebola virus infection to a subject.

Another embodiment is directed to a composition for at least preventing, treating, inhibiting, or attenuating an Ebola virus infection of a subject comprising a pharmaceutically acceptable carrier; and a tdsRNA.

In one aspect of any method, use, or composition of this disclosure, the composition may (1) not further comprise an active ingredient; (2) not further comprise an active ingredient that is an antigen; (3) not contain an antigen from the Ebola virus; (4) does not contain a nucleic acid with a sequence that is at least 90% identical to an Ebola virus nucleic acid; or (5) does not contain an Ebola virus nucleic acid.

In another aspect of any method, use, or composition of this disclosure, the composition further comprises at least one selected from the group consisting of: an absorption-promoting agent, a delivery-enhancing agent, a mucolytic agent, a mucus clearing agent, a ciliostatic agent, a penetration-promoting agent, a permeation-promoting agent, a vasodilator agent, a vasoconstrictor agent, RNase inhibitory agent, an enzyme inhibitor, a selective transport-enhancing agent, a stabilizing delivery vehicle, a carrier, a support, and a complex-forming species (antibody-antigen, avidin-biotin etc.).

In another aspect of any method, use, or composition of this disclosure, the subject is converted from seronegative for Ebola virus (i.e., no detectable antibodies to Ebola virus) to seropositive for Ebola (i.e., the presence of antibodies to Ebola virus can be detected) after exposure to Ebola virus without symptoms, or without the severe symptoms, of Ebola virus infection.

In another aspect of any method, use, or composition of this disclosure, immune resistance is produced in the subject after subsequent exposure to Ebola virus. The immune resistance may be, for example, immunity to a subsequent exposure to Ebola virus.

In another aspect of any method, use, or composition of this disclosure, the method produces immune resistance to Ebola virus infection is produced in the subject after exposure to Ebola virus—that is, after the initial exposure to the Ebola virus. In one aspect, the immune resistance to Ebola virus infection may persist for at least 10 days, at least 20 days, at least 30 days, at least 40 days, at least 50 days, at least 2 months, at least 3 months, at least 4 months, at least 6 months, at least 1 year, or at least 2 years.

In another aspect of any method, use, or composition of this disclosure, the composition may further comprise a natural mixture of human alpha interferons.

In another aspect of any method, use, or composition of this disclosure, the subject may be a mammal, a human, or a nonhuman animal.

In another aspect of any method, use, or composition of this disclosure, the tdsRNA may be selected from the group consisting of rIₙ•r(C₄₋₂₉U)ₙ; rIₙ•r(C₁₁₋₁₄U)ₙ; rIₙ•r(C₄U)ₙ; rIₙ•r(CsU)ₙ; rIₙ•r(C₆U)ₙ; rIₙ•r(C₇U)ₙ; rIₙ•r(C₈U)ₙ; rIₙ•r(C₉U)ₙ; rIₙ•r(C₁₀U)ₙ; rIₙ•r(C₁₁U)ₙ; rIₙ•r(C₁₂U)ₙ; rIₙ•r(C₁₃U)ₙ; rIₙ•r(C₁₄U)ₙ; rIₙ•r(C₁₅U)ₙ; rIₙ•r(C₁₆U)ₙ; rIₙ•r(C₁₇U)ₙ; rIₙ•r(C₁₈U)ₙ; rIₙ•r(C₁₉U)ₙ; rIₙ•r(C₂₀U)ₙ; rIₙ•r(C₂₁U)ₙ; rIₙ•r(C₂₂U)ₙ; rIₙ•r(C₂₃U)ₙ; rIₙ•r(C₂₄U)ₙ; rIₙ•r(C₂₅U)ₙ; rIₙ•r(C₂₆U)ₙ; rIₙ•r(C₂₇U)ₙ; rIₙ•r(C₂₈U)ₙ; rIₙ•r(C₂₉U)ₙ; rIₙ•r(C₃₀U)ₙ; rIₙ•r(C₃₁U)ₙ; rIₙ•r(C₃₂U)ₙ; rIₙ•r(C₃₃U)ₙ; rIₙ•r(C₃₄U)ₙ; rIₙ•r(C₃₅U)ₙ; rIₙ•r(C₄₋₃₀U)ₙ; rIₙ•r(C₁₄₋₃₀U)ₙ; rIₙ•r(C₁₁₋₁₄G)ₙ; rIₙ•r(C_{4- 29}G)ₙ; rIₙ•r(C₃₀₋₃₅U)ₙ; r(Poly I•Poly C)ₙ; r(Poly A•Poly U)ₙ; and any combination thereof.

In another aspect of any method, use, or composition of this disclosure, the tdsRNA is a rugged dsRNA that is resistant to denaturation under conditions that are able to separate hybridized poly(riboinosinic acid) and poly(ribocytosinic acid) strands (rIₙ•rCₙ).

In another aspect of any method, use, or composition of this disclosure, the length of the tdsRNA or n may be selected from the group consisting of: 40 to 50,000; 50 to 10,000; 60 to 9000; 70 to 8000; 80 to 7000; 40-500; 380 to 450; and any combination thereof.

In another aspect of any method, use, or composition of this disclosure, the tdsRNA may comprise 1 mol % to 4 mol % rugged dsRNA or 4 mol % to 16 mol % rugged dsRNA.

In another aspect of any method, use, or composition of this disclosure, the tdsRNA may comprise rIₙ•r(C₁₁₋₁₄U)ₙ and rugged dsRNA; or rIₙ•r(C₁₂U)ₙ and rugged dsRNA. In this aspect, the rugged dsRNA may have a formula of rIₙ•r(C₄₋₂₉U)ₙ, rIₙ•r(C₁₁₋₁₄U)ₙ, rIₙ•r(C₁₂U)ₙ, rIₙ•r(C₃₀U)ₙ, or rIₙ•r(C₃₀₋₃₅U)ₙ.

In another aspect of any method, use, or composition of this disclosure, the rugged dsRNA has one or more properties selected from the group consisting of: 40-500 bp in length; 380-450 bp in length; 250 kDa to 320 kDa in molecular weight; 30-38 dsRNA helical turns in length; formula of rIₙ•r(C₄₋₂₉U)ₙ; formula of rIₙ•r(C₁₁₋₁₄U)ₙ; formula of rIₙ•r(C₁₂U)ₙ; formula of rIₙ•r(C₃₀U)ₙ; and formula of rIₙ•r(C₃₀₋₃₅U)ₙ.

In another aspect of any method, use, or composition of this disclosure, the tdsRNA may have one or more physical properties selected from the group consisting of: about 4 to about 5000 helical turns of duplexed RNA; 30-38 helical turns of duplexed RNA; about 2 kilodaltons to about 30,000 kilodaltons molecular weight; and about 250 kilodaltons to about 320 kilodaltons molecular weight.

In another aspect of any method, use, or composition of this disclosure, the tdsRNA may have one or more of the following properties: at least 30 weight percent of total dsRNA in the composition is a linear structure; at least 40 weight percent of total dsRNA in the composition is a linear structure; at least 50 weight percent of total dsRNA in the composition is a linear structure; at least 60 weight percent of total dsRNA in the composition is a linear structure; at least 70 weight percent of total dsRNA in the composition is a linear structure; at least 80 weight percent of total dsRNA in the composition is a linear structure; or at least 90 weight percent of total dsRNA in the composition is a linear structure.

In another aspect of any method, use, or composition of this disclosure, the tdsRNA may be with a stabilizing polymer. For example, the stabilizing polymer is selected from the group consisting of polylysine; polylysine plus carboxymethylcellulose; polyarginine; polyarginine plus carboxymethylcellulose; carboxymethylcellulose; and any combination thereof.

In another aspect of any method, use, or composition of this disclosure, the composition is administered at a dosage of about 25-700 milligrams of tdsRNA.

In another aspect of any method, use, or composition of this disclosure, the composition is administered at a rate which is selected from the group consisting of: one dose per day, one dose every 2 days, one dose every 3 days, one dose every 4 days, one dose every 5 days, once a week, twice a week, 3 times a week, once every two weeks, once every 3 weeks, once every 4 weeks, and once a month.

In another aspect of any method, use, or composition of this disclosure, the composition the natural mixture of human alpha interferons used is a purified mixture of at least three different human interferon-alpha proteins with native amino acid sequences and glycosylation patterns, preferably the natural mixture of human alpha interferons is ALFERON N Injection® (Interferon Alfa-N3).

In another aspect of any method, use, or composition of this disclosure, the composition, where the natural mixture of human alpha interferons used, it is administered in a dosage from 5 IU per pound body weight/day to 100,000 IU per pound body weight/day.

In another aspect of any method, use, or composition of this disclosure, the administering is at least one selected from the group consisting of: systemic administration; intravenous administration; intradermal administration; subcutaneous administration; intramuscular administration; nasal administration (pulmonary airway administration); intraperitoneal administration; intracranial administration; intravesical administration; oral administration (through the mouth, by breathing through the mouth); intravaginal administration, intrarectal administration, intratracheal administration, oropharyngeal administration, sublingual administration, topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; dry powder inhalants administration; and any combination thereof.

In another aspect of any method, use, or composition of this disclosure, the administering is by a delivery system (device) selected from the group consisting of: a nebulizer; a sprayer; a nasal pump; a squeeze bottle; a nasal spray; a syringe sprayer or plunger sprayer (a syringe providing pressure to an attached sprayer or nozzle); a nasal aerosol device; a controlled particle dispersion device; a nasal aerosol device; a nasal nebulization device; a pressure-driven jet nebulizer; ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered-dose inhalers; a dry powder inhalation devices; an instillation device; an intranasal instillation device; an intravesical instillation device; a swab; a pipette; a nasal irrigation device; a nasal rinse; an aerosol device; a metered aerosol device; a pressurized dosage device; a powdered aerosol; a spray aerosol; a spray device; a metered spray device; a suspension spray device; and any combination thereof.

In another aspect of any method, use, or composition of this disclosure, the composition is a prophylactic or therapeutic vaccine, wherein the vaccine comprises one or more Ebola antigens or at least an inactivated or attenuated Ebola virus. The Ebola virus antigen may be an antigen purified from an Ebola virus or an inactivated Ebola virus.

In another aspect of any method, use, or composition of this disclosure, the composition is a nasal vaccine.

In another aspect of any method, use, or composition of this disclosure, a combination of the tdsRNA and the Ebola antigen may provide a vaccine effect that is superior to that of the Ebola antigen administered alone. Superior vaccine effect would include a longer immunity, a stronger immunity against, for example, a higher titer of Ebola virus infection, a faster establishment of immunity, a reduction in the severity of an Ebola infection, a reduction in side effects due to the vaccine or to Ebola infection.

### DETAILED DESCRIPTION

Not wishing to be bound by any theory or mechanism or action, there is a rationale for our early drug intervention. Studies of viral pathogenesis have clearly demonstrated that the first step in pathogenesis is entry of virus into the host subject. One of the main routes of entry in humans is via the respiratory tract. The respiratory tract is populated with epithelial cells and dendritic cells. Epithelial cells possess a variety of molecular surface structures, which may serve as cell receptors that interact with viral attachment proteins. Therefore, the nasal administration of medicament is especially preferred if the medicament can have an effect of preventing Ebola transmission. We have found that low levels of Ebola can cause Ebola virus infections. However, if viral infections and transmission can be stopped, infection of the host or the manifestation of serious symptoms may be prevented.

### Definitions

“r” and “ribo” has the same meaning and refer to ribonucleic acid or the nucleotide or nucleoside that are the building block of ribonucleic acid.

RNA consists of a chain of linked units called nucleotides. Unless otherwise specified, the nucleotides and bases expressed refers to the ribo form of the nucleotide or base (i.e., ribonucleotide with one or more phosphate groups). Therefore “A” refers to rA or adenine, “U” refers to rU or uracil, “C” refers to rC or cytosine, “G” refers to rG or guanine, “I” refers to rI or inosine, “rN” refers to rA, rU, rC, rG or rI. Each of these (i.e., A, U, C, G, I) may have one or more phosphate groups as discussed above.

“n” is a positive number and refers to the length of the ssRNA or dsRNA in bases or basepairs. “n” can be a positive integer when referring to one nucleic acid or it can be any positive number when it is an average length of a population of nucleic acids.

Single-stranded RNA or double-stranded RNA, may have a ratio of nucleotides or bases. For example, r(C₁₂U)ₙ denotes a single RNA strand that has, on average 12 C bases or nucleotides for every U base or nucleotide. As another example, r(C₁₁₋₁₄U)ₙ denotes a single RNA strand that has, on average 11 to 14 C bases or nucleotides for every U base or nucleotide. As another example, the formula “rIₙ•r(C₁₁₋₁₄U)ₙ” refers to a double-stranded RNA, one strand is poly(I) and the second strand is r(C₁₁₋₁₄U)ₙ.

As an example, the formula “rIₙ•r(C₁₂U)ₙ” can be expressed as “riboIₙ•ribo(C₁₂U)ₙ”, “rIₙ•ribo(C₁₂U)ₙ”, or “riboIₙ•r(C₁₂U)ₙ”. It refers to a double-stranded RNA with two strands. One strand (rIₙ) is poly ribo-inosine of n bases in length. The other strand is ssRNA of random sequence of C and U bases, the random sequence ssRNA is n bases in length, and a ratio of C bases to U bases in the random sequence ssRNA is about 12 (i.e., mean 12 C to 1 U).

The “•” symbol indicates that one strand of the dsRNA is hybridized (hydrogen-bonded) to the second strand of the same dsRNA. Therefore, rIₙ•r(C₁₂U)ₙ is double-stranded RNA comprising two ssRNA. One ssRNA is poly(I) (or rIₙ) and the other ssRNA is poly(C₁₂U) (or r(C₁₂U)ₙ). It should be noted that while we referred to the two strands being hybridized, not 100% of the bases form base pairing as there are some bases that are mismatches. Also, because rU does not form base pairing with rI as well as rC form base paring with rI, rU provides a focus of hydrodynamic instability in rIₙ•r(C₁₂U)ₙ at the locations of the U bases.

As discussed earlier, the term “r” and “ribo” has the same meaning in the formulas of the disclosure. Thus, as an example, rI, riboI, r(I), and ribo(I) refer to the same chemical which is the ribose form of inosine. Similarly, rC, riboC, r(C), and ribo(C) all refer to cytidine in the ribose form which is a building block of RNA. rU, riboU, r(U) and ribo(U) all refer to Uracil in the ribose form, which is a building block of RNA.

In this disclosure, inosine is also considered a possible rNMP, rNDP or rNTP. Inosine is a nucleoside that is formed when hypoxanthine is attached to a ribose ring (also known as a ribofuranose) via a β-N9-glycosidic bond.

In a preferred embodiment, the tdsRNA may comprises between 0.1% to 4% ssRNA, between 0.5% to 3% ssRNA, and preferably between 1.5% to 2.5% ssRNA.

While this disclosure refers to dsRNA and tdsRNA, it is not required that the tdsRNA comprising only two ssRNA in duplex. For example, tdsRNA may comprise one strand of 300 bases and (1) two opposite strands of 150 bases each, or three opposite strands of 100 bases each.

The dsRNA (tdsRNA) and ssRNA of this disclosure are different and distinct from mRNA. For example, the ssRNA and dsRNA (tdsRNA) of this disclosure are preferably missing one or all of the following which are associated with mRNA: (1) 5′ cap addition, (2) polyadenylation, (3) start codon, (4) stop codon, heterogeneous protein-coding sequences, and (5) spice signals.

The terms “intranasal” or “intranasally,” “instillation,” “instillation of a liquid,” “instillation using a sprayer” as used herein, refers to a route of delivery of an active compound to a patient by inhalation to the nasal mucosa, the airway, the lung or a combination thereof.

Unless otherwise specified, the term “Ebola” should be considered to be the equivalent of “Ebola virus.” Therefore, for example, “Ebola infection” refers to Ebola virus infection.

tdsRNA

The double-stranded RNAs described in this disclosure are therapeutic double-stranded “tdsRNA” which has a number of benefits when administered either by itself or with other medicaments and pharmaceuticals to a subject. In one aspect, the “tdsRNA” which can serve in a therapeutic capacity as well as in a preventative capacity against Ebola virus infection. All of the tdsRNAs of this disclosure are designed to reduce the Ebola viral load and/or prevent or at least reduce the risk of Ebola virus infection of a susceptible individual. In other aspects, the tdsRNA has antiviral effects, or an adjuvant effect when administered with a vaccine. tdsRNA includes, at least, AMPLIGEN® (rintatolimod, which is a tdsRNA of the formula rIₙ•r(C₁₂U)ₙ). tdsRNA can be supplied as a solution in Phosphate Buffered Saline (PBS).

tdsRNA Structural Definition Another aspect is directed to a tdsRNA produced by any of the methods of this disclosure—referred to herein as the “tdsRNA Product” or “tdsRNA”—the two terms have the same meaning.

The tdsRNA may be at least one selected from the group consisting of: rIₙ•r(C₄U)ₙ, rIₙ•r(C₅U)ₙ, rIₙ•r(C₆U)ₙ, rIₙ•r(C₇U)ₙ, rIₙ•r(C₈U)ₙ, rIₙ•r(C₉U)ₙ, rIₙ•r(C₁₀U)ₙ, rIₙ•r(C₁₁U)ₙ, rIₙ•r(C₁₂U)ₙ, rIₙ•r(C₁₃U)ₙ, rIₙ•r(C₁₄U)ₙ, rIₙ•r(C₁₅U)ₙ, rIₙ•r(C₁₆U)ₙ, rIₙ•r(C₁₇U)ₙ, rIₙ•r(C₁₈U)ₙ, rIₙ•r(C₁₉U)ₙ, rIₙ•r(C₂₀U)ₙ, rIₙ•r(C₂₁U)ₙ, rIₙ•r(C₂₂U)ₙ, rIₙ•r(C₂₃U)ₙ, rIₙ•r(C₂₄U)ₙ, rIₙ•r(C₂₅U)ₙ, rIₙ•r(C₂₆U)ₙ, rIₙ•r(C₂₇U)ₙ, rIₙ•r(C₂₈U)ₙ, rIₙ•r(C₂₉U)ₙ, rIₙ•r(C₃₀U)ₙ, rIₙ•r(C₃₁U)ₙ, rIₙ•r(C₃₂U)ₙ, rIₙ•r(C₃₃U)ₙ, rIₙ•r(C₃₄U)ₙ, rIₙ•r(C₃₅U)ₙ, rIₙ•r(C₄₋₂₉U)ₙ, rIₙ•r(C₁₁₋₁₄U)ₙ, rIₙ•r(C₃₀₋₃₅U)ₙ, rIₙ•r(C₄₋₂₉G)ₙ, rIₙ•r(C₂₀G)ₙ, rIₙ•r(C₂₉G)ₙ, and rIₙ•r(AU)ₙ.

Where there is no subscript denoting length or ratio, the default value is “1.” For example, rIₙ•r(C₁₂U)ₙ is the same as rIₙ•r(C₁₂U₁)ₙ. The length of the tdsRNA is denoted as a lowercase “n” (e.g., rIₙ•r(C₁₂U)ₙ).

In another aspect, at least 70%, at least 80%, or at least 90% of the tdsRNA may have a molecular weight of between 400,000 Daltons to 2,500,000 Daltons. The value of 70 percent in the previous sentence may be weight percent or molar percent.

In another aspect, the tdsRNA comprises a first ssRNA and a second ssRNA and each of these first ssRNA or second ssRNA may contain one or more strand breaks.

In another aspect, the tdsRNA may comprise at least one selected from the group consisting of: a 3′ overhang, a 5′ overhang, a blunt end, an internal ssRNA sequence, one or more strand breaks in a first ssRNA, and one or more strand breaks in a second ssRNA.

In another aspect, the tdsRNA is a linear molecule—that is a molecule that is not branched or that does not contain any loop structure. In different aspects, at least 60%, at least 70%, at least 80%, at least 90%, at least 95% or 100% of the tdsRNA is a linear molecule.

In another aspect, the tdsRNA has the property that greater than about 90%, greater than 95%, greater than 98%, greater than 99%, or 100% of the bases of the RNA are in a double-stranded configuration.

Another aspect is directed to a therapeutic composition comprising: a tdsRNA, and a pharmaceutically acceptable excipient.

One embodiment is directed to rintatolimod, which is a tdsRNA of the formula rIₙ•r(C₁₂U)ₙ and which is also denoted by the trademark AMPLIGEN®. rIₙ•r(C₁₂U)ₙ is a synthetic double-stranded ribonucleic acid in which uridylic acid (U) substitution in the cytidylic chain creates a region of non-hydrogen bonding with the rIₙ chain in molecular configuration. The chemical name for this embodiment of tdsRNA is polyriboinosinic:polyribocytidylic(12:1)uridylic acid which can be expressed as: Poly I:Poly C₁₂U or rIₙ•r(C₁₂U)ₙ.

In one embodiment, the tdsRNA comprises mismatched dsRNA such as an RNA strand comprising riboinosinic acid and an RNA strand comprising ribocytidylic acid and ribouracilic acid. This can be expressed as rIₙ•r(CₓU)ₙ. where “x” is a positive number or a range of positive numbers. Examples of X include 11, 12, 13, 14, 11-14, 4-29, 4-30, 4-35 and combinations thereof.

In a preferred embodiment, the tdsRNA are of the general formula rIₙ•r(C₁₁₋₁₄, U)ₙ and are described in U.S. Pat. Nos. 4,024,222 and 4,130,641 (which are incorporated by reference herein) or synthesized according to this disclosure.

In one embodiment, the tdsRNA comprises mismatched dsRNA such as an RNA strand comprising riboinosinic acid and an RNA strand comprising ribocytosinic acid and guanine. This can be expressed as rIₙ•r(CₓG)ₙ. where “x” is a positive number or a range of positive numbers (including fractions). Examples of X include 11, 12, 12.5, 13, 13.5 14, 11-14, and 4-35 and a preferred value of x is 12.

In one embodiment, the tdsRNA is matched RNA rAₙ•rUₙ. That is, in this case, the tdsRNA may be matched (i.e., not in mismatched form). Thus, polyadenylic acid complexed with polyuridylic acid (i.e., (rA•rU)ₙ) may be used. The matched dsRNA may be administered in the same method as any of the mismatched tdsRNAs.

Length

The length of the tdsRNA, which is also represented in formulas as “n,” can be measured in basepairs. Other units of length or size commonly used by one of ordinary skill in the art include molecular weight or the number of turns of a double-stranded RNA structure. For example, it is generally accepted that there are about 629 daltons per base pair. Therefore, by knowing one of three parameters which are (1) length in bps (basepairs), (2) molecular weight (e.g., in Daltons or kiloDaltons (kDa)) of both strands, or (3) the number of turns of dsRNA (or any nucleic acid such as dsDNA), the other two parameters can be easily calculated by one of ordinary skill in the art. Unless otherwise defined in this disclosure, it is understood that the “number of turns of nucleic acid” or “the number of helical turns” refers to dsRNA. The length of tdsRNA can therefore be selected from the group consisting of: 4 bps to 5000 bps, 10 bps to 50 bps, 10 bps to 500 bps, 10 bps to 40,000 bps, 40 bps to 40,000 bps, 40 bps to 50,000 bps, 40 bps to 500 bps, 50 bps to 500 bps, 100 bps to 500 bps, 380 bps to 450 bps, 400 bps to 430 bps, 30 kDa to 300 kDa molecular weight, 250 kDa to 320 kDa molecular weight, 270 kDa to 300 kDa molecular weight, 4.7 to 46.7 helical turns of duplexed RNA, 30 to 38 helical turns of duplexed RNA, 32 to 36 helical turns of duplexed RNA, and a combination thereof. The tdsRNA may be a combination of lengths where, for example, the tdsRNA is a combination of different populations of tdsRNA sizes. The length may be an average basepair, average molecular weight, or an average helical turns of duplexed RNA and can take on the value of any number (e.g., integer or fraction).

Rugged dsRNA

Rugged dsRNA is a tdsRNA that is resistant to denaturation under conditions that are able to separate hybridized poly(riboinosinic acid) and poly(ribocytosinic acid) strands (that is, rIₙ•rCₙ strands). See, U.S. Pat. Nos. 8,722,874 and 9,315,538 (incorporated by reference) for a further description of Rugged dsRNA and exemplary methods of preparing such molecules.

In one aspect, a rugged dsRNA can be an isolated double-stranded ribonucleic acid (dsRNA) which is resistant to denaturation under conditions that are able to separate hybridized poly(riboinosinic acid) and poly(ribocytosinic acid) strands, wherein only a single strand of said isolated dsRNA comprises one or more uracil or guanine bases that are not base-paired to an opposite strand and wherein said single strand is comprised of poly(ribocytosinic₃₀₋₃₅uracilic acid). Further, the single strand may be partially hybridized to an opposite strand comprised of poly(riboinosinic acid). In another aspect, rugged dsRNA may be an isolated double-stranded ribonucleic acid (dsRNA) which is resistant to denaturation under conditions that are able to separate hybridized poly(riboinosinic acid) and poly(ribocytosinic acid) strands.

In another aspect, Rugged dsRNA, has at least one of the following: r(Iₙ)·r(C₄₋₂₉U)ₙ, r(Iₙ)•r(C₁₂U)ₙ,r(Iₙ)•r(C₁₁₋₁₄U)ₙ, r(Iₙ)•r(C₁₂U)ₙ, r(Iₙ)•r(C₃₀U)ₙ, or r(Iₙ)•r(C₃₀₋₃₅U)ₙ. In another aspect, Rugged dsRNA may have a size of 4 bps to 5000 bps, 40 bps to 500 bps, 50 bps to 500 bps, 380 bps to 450 bps, 400 bps to 430 bps, 30 kDa to 300 kDa molecular weight, 250 kDa to 320 kDa molecular weight, 270 kDa to 300 kDa molecular weight, 4.7 to 46.7 helical turns of duplexed RNA, 30 to 38 helical turns of duplexed RNA, 32 to 36 helical turns of duplexed RNA, and a combination thereof.

In another aspect, Rugged dsRNA is produced by isolating the 5 minute HPLC peak of a tdsRNA preparation.

Rugged dsRNA Preparation

In one embodiment, the starting material for making Rugged dsRNA may be dsRNA prepared in vitro using conditions of this disclosure. For example, the specifically configured dsRNA described in U.S. Pat. Nos. 4,024,222, 4,130,641, and 5,258,369 (which are incorporated by reference herein) are generally suitable as starting materials after selection for rugged dsRNA. tdsRNA (or preparations of tdsRNA) described in this disclosure is also useful as starting material.

After procuring starting material, Rugged dsRNA may be isolated by at least subjecting the partially hybridized strands of a population of dsRNA to conditions that denature most dsRNA (more than 10 wt % or mol %, more than 20 wt % or mol %, more than 30 wt % or mol %, more than 40 wt % or mol %, more than 50 wt % or mol %, more than 60 wt % or mol %, more than 70 wt % or mol %, more than 80 wt % or mol %, more than 90 wt % or mol %, more than 95 wt % or mol %, or more than 98 wt % or mol %) in the population, and then selection negatively or positively (or both) for dsRNA that remain partially hybridized. The denaturing conditions to unfold at least partially hybridized strands of dsRNA may comprise an appropriate choice of buffer salts, pH, solvent, temperature, or any combination thereof. Conditions may be empirically determined by observation of the unfolding or melting of the duplex strands of ribonucleic acid. The yield of rugged dsRNA may be improved by partial hydrolysis of longer strands of ribonucleic acid, then selection of (partially) hybridized stands of appropriate size and resistance to denaturation.

The purity of rugged dsRNA, which functions as tdsRNA, may thus be increased from less than about 0.1-10 mol % (e.g., rugged dsRNA is present in at least 0.1 mol % or 0.1 wt percent but less than about 10 mol % or 10 wt percent) relative to all RNA in the population after synthesis to a higher purity. A higher purity may be more than 20 wt % or mol %, more than 30 wt % or mol %, more than 40 wt % or mol %, more than 50 wt % or mol %, more than 60 wt % or mol %, more than 70 wt % or mol %, more than 80 wt % or mol %, more than 90 wt % or mol %, more than 98 wt % or mol %, or between 80 to 98 wt % or mol %. All wt % or mol % is relative to all RNA present in the same composition.

Another method of isolating Rugged dsRNA is to employ chromatography. Under analytical or preparative high-performance liquid chromatography, Rugged dsRNA can be isolated from a preparation (e.g., the starting material as described above) to produce poly(I):poly(C₁₂U), (e.g., poly(I):poly(C₁₁₋₁₄U)ₙ) as a substantially purified and pharmaceutically-active molecule with an HPLC peak of about 4.5 to 6.5 minutes, preferably between 4.5 and 6 minutes and most preferably 5 minutes.

### Comments Regarding All Embodiments

For any of the embodiments, the numeric subscript of the formulas can be seen as a ratio of the bases. For example, in the formula rIₙ•r(C₁₁₋₁₄U)ₙ the ratio between two types of bases (i.e., C and U in this case) is 11 to 14 and any value in between because the value 11-14 is an average ratio of a population of nucleic acids. Similarly, n can be any positive number because it is an average length. The values of n is discussed in other parts of this disclosure.

Stabilizing Polymers

In any of the described embodiments, the tdsRNA may be complexed with a stabilizing polymer such as: polylysine, polylysine plus carboxymethylcellulose (lysine carboxy methyl cellulose), polyarginine, polyarginine plus carboxymethylcellulose, or a combination thereof. Some of these stabilizing polymers are described, for example, in U.S. Pat. No. 7,439,349.

Modified Backbone

The tdsRNA may comprise one or more alterations in the backbone of the nucleic acid. For example, configured tdsRNA may be made by modifying the ribosyl backbone of poly(riboinosinic acid) r(Iₙ), for example, by including 2′-O-methylribosyl residues. Specifically configured dsRNA may also be modified at the molecule's ends to add a hinge(s) to prevent slippage of the base pairs, thereby conferring specific bioactivity in solvents or aqueous environments that exist in human biological fluids.

Additional Agents

Any agents or active ingredients including tdsRNA and a natural mixture of human alpha interferons can be combined in any manner with each other for any of the method, use, or composition of this disclosure.

The tdsRNA of this disclosure may be in a compound or in a combination with a number of additional agents. Examples of these agents are described herein.

Carrier or Vehicle

Suitable agents may include a suitable carrier or vehicle for intranasal mucosal delivery. As used herein, the term “carrier” refers to a pharmaceutically acceptable solid or liquid filler, diluent or encapsulating material. In one aspect, the carrier is a suitable carrier or vehicle for intranasal mucosal delivery including delivery to the air passages and to the lungs of a subject.

A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as, for example, lactose, glucose and sucrose, starches such as corn starch and potato starch, cellulose and its derivatives such as sodium carboxymethyl cellulose, ethyl cellulose and cellulose acetate, powdered tragacanth, malt, gelatin, talc, excipients such as cocoa butter and suppository waxes, oils such as peanut oil, cottonseed oil, safflower oil, sesame oil, olive oil, corn oil and soybean oil, glycols, such as propylene glycol, polyols such as glycerin, sorbitol, mannitol and polyethylene glycol, esters such as ethyl oleate and ethyl laurate, agar, buffering agents such as magnesium hydroxide and aluminum hydroxide, alginic acid, pyrogen free water, isotonic saline, Ringer's solution, ethyl alcohol and phosphate buffer solutions, phosphate buffered saline (PBS), Tris buffer solution, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants which can be administered with tdsRNA include water-soluble antioxidants such as ascorbic acid, cysteine hydrochloride, sodium bisulfite, sodium metabisulfite, sodium sulfite and the like, oil-soluble antioxidants such as ascorbyl palmitate, butylated hydroxyanisole (BHA), butylated hydroxytoluene (BHT), lecithin, propyl gallate, alpha-tocopherol and the like, and metal-chelating agents such as citric acid, ethylenediamine tetraacetic acid (EDTA), sorbitol, tartaric acid, phosphoric acid and the like.

Absorption-Promoting Agents

Suitable agents may include any suitable absorption-promoting agents. The suitable absorption-promoting agents may be selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacyl methyl sulfoxide, azone, sodium lauryl sulfate, oleic acid, and bile salts, may be employed to enhance mucosal penetration of the tdsRNA. In additional aspects, surfactants (e.g., polysorbates) are employed as adjunct compounds, processing agents, or formulation additives to enhance intranasal delivery of the tdsRNA.

Delivery-Enhancing Agents

As used herein, the term “delivery-enhancing agents” refers to any agents which enhance the release or solubility (e.g., from a formulation delivery vehicle), diffusion rate, penetration capacity and timing, uptake, residence time, stability, effective half-life, peak or sustained concentration levels, clearance and other desired intranasal delivery characteristics (e.g., as measured at the site of delivery, or at a selected target site of activity such as the bloodstream) of tdsRNA or other biologically active compound(s).

In one aspect, enhancement of intranasal delivery can thus occur by any of a variety of mechanisms, for example by increasing the diffusion, transport, persistence or stability of tdsRNA, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

Mucolytic or Mucus Clearing Agents

In another embodiment, the present formulations may also comprise other suitable agents such as mucolytic and mucus-clearing agents. The term “mucolytic and mucus-clearing agents,” as used herein, refers to any agents which may serve to degrade, thin or clear mucus from intranasal mucosal surfaces to facilitate absorption of intranasally administered biotherapeutic agents including tdsRNA. Based on their mechanisms of action, mucolytic and mucus clearing agents can often be classified into the following groups: proteases (e.g., pronase, papain) that cleave the protein core of mucin glycoproteins, sulfhydryl compounds that split mucoprotein disulfide linkages, and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine. Other effective agents that reduce mucus viscosity or adhesion to enhance intranasal delivery according to the methods of the disclosure include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Ciliostatic Agents

In another embodiment, the present formulations may comprise ciliostatic agents. As used herein, the term “ciliostatic agents” refers to any agents which are capable of moving a layer of mucus along the mucosa to removing inhaled particles and microorganisms. For use within these aspects of the disclosure, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of tdsRNA and other biologically active agents without unacceptable adverse side effects.

Within more detailed aspects, a specific ciliostatic factor may be employed in a combined formulation or coordinate administration protocol with tdsRNA, and/or other biologically active agents disclosed herein. Various bacterial ciliostatic factors isolated and characterized in the literature may be employed within these embodiments of the disclosure. Ciliostatic factors from the bacterium Pseudomonas aeruginosa include a phenazine derivative, a pyo compound (2-alkyl-4-hydroxyquinolines), and a rhamnolipid (also known as a hemolysin).

Penetration or Permeation-Promoting Agents

In another embodiment, the intranasal mucosal therapeutic and prophylactic formulations of the present disclosure may be supplemented with any suitable penetration-promoting agent that facilitates absorption, diffusion, or penetration of tdsRNA across mucosal barriers. The penetration promoter may be any promoter that is pharmaceutically acceptable. Thus, another aspect relates to compositions comprising tdsRNA and one or more penetration-promoting agents selected from sodium salicylate and salicylic acid derivatives (acetyl salicylate, choline salicylate, salicylamide, etc.), amino acids and salts thereof (e.g., monoaminocarboxlic acids such as glycine, alanine, phenylalanine, proline, hydroxyproline, etc., hydroxyamino acids such as serine, acidic amino acids such as aspartic acid, glutamic acid, etc., and basic amino acids such as lysine, etc.—inclusive of their alkali metal or alkaline earth metal salts), and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts).

Also provided as penetration-promoting agents within the methods and compositions of the disclosure are substances which are generally used as emulsifiers (e.g., sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidones carboxylic acid esters, N-alkylpyrrolidones, proline acyl esters, and the like.

In another embodiment, the present formulation may also comprise other suitable agents such as nitric oxide donor agents. As used herein, the term “nitric oxide donor agents” refers to any suitable agents which are capable of releasing nitric oxide. The release of nitric oxide may have a vasodilating effect. A nitric oxide (NO) donor may be selected as a membrane penetration-enhancing agent to enhance mucosal delivery of tdsRNA, and other biologically active agents disclosed herein. Various NO donors are known in the art and are useful in effective concentrations within the methods and formulations of the disclosure. Exemplary NO donors include, but are not limited to, nitroglycerine, nitroprusside, NOC5 [3-(2-hydroxy-1-(methyl-ethyl)-2-nitrosohydrazino)-1-propanamine], NOC12 [N-ethyl-2-(1-ethyl-hydroxy-2-nitrosohydrazino)-ethanamine], SNAP [S-nitroso-N-acetyl-DL-penicillamine], NORI and NOR4. Within the methods and compositions of the disclosure, an effective amount of a selected NO donor may be coordinately administered or combinatorically formulated with tdsRNA, and/or other biologically active agents disclosed herein, into or through the mucosal epithelium.

Non-limiting examples of other permeation enhancers useful in the instant disclosure are the simple long-chain esters that are Generally Recognized As Safe (GRAS) in the various pharmacopoeial compendia. These may include simple aliphatic, unsaturated or saturated (but preferably fully saturated) esters, which contain up to medium length chains. Non-limiting examples of such esters include isopropyl myristate, isopropyl palmitate, myristyl myristate, octyl palmitate, and the like. The enhancers are of a type that are suitable for use in a pharmaceutical composition. The artisan of ordinary skill will also appreciate that those materials that are incompatible with or irritating to mucous membranes should be avoided.

For nasal administration, the enhancer is present in the composition in a concentration effective to enhance penetration of the pharmaceutically active agent that is to be delivered through the nasal mucosa. Various considerations should be taken into account in determining the amount of enhancer to use. Such considerations include, for example, the amount of flux (rate of passage through the membrane) achieved and the stability and compatibility of the components in the formulations. The enhancer is generally used in an amount of about 0.001 to about 40 (w/w) % of the composition. Specific ranges include, about 0.01% to about 30 (w/w), about 0.1 to about 25% (w/w), about 1% to about 15% (w/w), about 5 to 10% (w/w). Alternatively, the amount of the enhancer may range from about 1.0 to about 3% (w/w) or about 10 to about 20% (w/w).

Any of the above permeation enhancers are useful, especially in nasal administration.

Vasodilator or Vasoconstrictor Agents

In another embodiment, the present formulation may also comprise other suitable agents such as vasodilator agents. As used herein, the term “vasodilator agents” refers to any agents which are vasoactive. A vasodilator agent may function within the disclosure to modulate the structure and physiology of the submucosal vasculature, increasing the transport rate of tdsRNA, and other biologically active agents into or through the mucosal epithelium and/or to specific target tissues or compartments (e.g., the systemic circulation). Vasodilator agents for use within the disclosure typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, alpha-adrenergic and imidazole receptor antagonists, beta-1-adrenergic agonists, phosphodiesterase inhibitors, eicosanoids and NO donors.

In another embodiment, the present formulation may also comprise other suitable agents such as vasoconstrictor agents. As used herein, the term “vasoconstrictor agents” refers to any substances which may cause vasoconstriction. Vasoconstrictor agents may usually cause an increase in systemic blood pressure, but when they are administered in specific tissues, localized blood flow may be reduced. Vasoconstrictor agents may include any suitable substances such as antihistamines, decongestants and stimulants that are used to treat ADHD.

RNase Inhibitory Agents and Enzyme Inhibitors

In some embodiments, for example, nasal vaccines, the disclosure encompasses the delivery of a protein, peptide or other nucleic acid in addition to tdsRNA. Therefore, the compositions of the present disclosure may contain an enzyme inhibitor. As is well known to practitioners in nucleic acid, peptide and protein biochemistry, these biopolymers tend to be very sensitive to the presence of enzymes, such as RNase and proteolytic enzymes, that rapidly degrade the biopolymer when present in even minute amounts. Typical enzyme inhibitors that are commonly employed and that may be incorporated into the present disclosure include, but are not limited to leupeptin, aprotinin, and the like. Enzyme inhibitors also include nuclease inhibitors such as DNase inhibitors and RNase inhibitors. RNase inhibitors are commonly used as a precautionary measure in enzymatic manipulations of RNA to inhibit and control RNase. These are commercially available from a number of sources such as, for example, Invitrogen (SUPERase, In RNase Inhibitor, RNaseOUT, RNAsecure, and RNase Inhibitor).

Selective Transport-Enhancing Agents

In another embodiment, the present formulation may also comprise other suitable agents such as selective transport-enhancing agents. As used herein, the term “selective transport-enhancing agent” refers to any agent that facilitates transport of tdsRNA and/or one or more biologically active agents including vaccines. The compositions and delivery methods of the disclosure may optionally incorporate a selective transport-enhancing agent that facilitates transport of one or more biologically active agents. These transport-enhancing agents may be employed in a combinatorial formulation or coordinate administration protocol with tdsRNA disclosed herein, to coordinately enhance delivery of one or more additional biologically active agent(s). Alternatively, the transport-enhancing agents may be employed in a combinatorial formulation or coordinate administration protocol to directly enhance mucosal delivery of tdsRNA, with or without enhanced delivery of an additional biologically active agent.

Exemplary selective transport-enhancing agents for use within this aspect of the disclosure may include, but are not limited to, glycosides, sugar-containing molecules, and binding agents such as lectin binding agents, and stabilizers. For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within the disclosure. Certain bioadhesive ligands for use within the disclosure will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more tdsRNA or functionally equivalent fragment proteins, analogs and mimetics, and other biologically active agent(s) into and/or through mucosal epithelia. These and other selective transport-enhancing agents significantly enhance mucosal delivery of macromolecular biopharmaceuticals (particularly peptides, proteins, oligonucleotides and polynucleotide vectors) within the disclosure.

Additional intranasal mucosal delivery-enhancing agents that are useful within the coordinated administration and processing methods and combinatorial formulations of the disclosure may also include, but are not limited to, mixed micelles, enamines, nitric oxide donors (e.g., S-nitroso-N-acetyl-DL-penicillamine, NOR1, NOR4—which are preferably co-administered with a nitric oxide scavenger such as carboxy-PITO or diclofenac sodium), sodium salicylate, glycerol esters of acetoacetic acid (e.g., glyceryl-1,3-diacetoacetate or 1,2-isopropylideneglycerine-3-acetoacetate), and other release-diffusion or intra- or trans-epithelial penetration-promoting agents that are physiologically compatible for intranasal mucosal delivery. Other absorption-promoting agents may be selected from a variety of carriers, bases and excipients that enhance mucosal delivery, stability, activity or trans-epithelial penetration of the tdsRNA. These include, inter alia, cyclodextrins and beta-cyclodextrin derivatives (e.g., 2-hydroxypropyl-beta-cyclodextrin and heptakis(2,6-di-O-methyl-beta-cyclodextrin). These compounds, optionally conjugated with one or more of the active ingredients and further optionally formulated in an oleaginous base, enhance bioavailability in the intranasal mucosal formulations. Yet additional absorption-enhancing agents adapted for intranasal mucosal delivery may also include medium-chain fatty acids, including mono- and diglycerides (e.g., sodium caprate—extracts of coconut oil, CAPMUL), and triglycerides (e.g., amylodextrin, Estaram 299, Miglyol 810).

Stabilizing Delivery Vehicle, Carrier, Support or Complex-Forming Species

In another embodiment, the present formulation may also comprise other suitable agents such as a stabilizing delivery vehicle, carrier, support or complex-forming species. The coordinate administration methods and combinatorial formulations of the instant disclosure may optionally incorporate effective lipid or fatty acid-based carriers, processing agents, or delivery vehicles, to provide improved formulations for mucosal delivery of tdsRNA or functionally equivalent fragment proteins, analogs and mimetics, and other biologically active agents. For example, formulations and methods for mucosal delivery can comprise one or more of these active agents, such as a peptide or protein, admixed or encapsulated by, or coordinately administered with, a liposome, mixed micellar carrier, or emulsion, to enhance chemical and physical stability and increase the half-life of the biologically active agents (e.g., by reducing susceptibility to proteolysis, chemical modification and/or denaturation) upon mucosal delivery.

Within certain aspects of the disclosure, specialized delivery systems for biologically active agents may comprise small lipid vesicles known as liposomes or micelles. These are typically made from natural, biodegradable, non-toxic, and non-immunogenic lipid molecules, and can efficiently entrap or bind drug molecules, including peptides and proteins, into, or onto, their membranes. The attractiveness of liposomes as a nucleic acid delivery system is increased by the fact that the encapsulated tdsRNA can remain in their preferred aqueous environment within the vesicles, while the liposomal membrane protects them against nuclease and other destabilizing factors.

Additional delivery vehicles carrier, support or complex-forming species for use within the disclosure may include long and medium-chain fatty acids, as well as surfactant mixed micelles with fatty acids. Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the disclosure, long-chain fatty acids, especially fusogenic lipids (unsaturated fatty acids and monoglycerides such as oleic acid, linoleic acid, linoleic acid, monoolein, etc.) provide useful carriers to enhance mucosal delivery of tdsRNA, and other biologically active agents disclosed herein. Medium-chain fatty acids (C6 to C12) and monoglycerides have also been shown to have enhancing activity in intestinal drug absorption and can be adapted for use within the mucosal delivery formulations and methods of the disclosure. In addition, sodium salts of medium and long-chain fatty acids are effective delivery vehicles and absorption-enhancing agents for mucosal delivery of biologically active agents. Thus, fatty acids can be employed in soluble forms of sodium salts or by the addition of non-toxic surfactants, e.g., polyoxyethylated hydrogenated castor oil, sodium taurocholate, etc. Other fatty acid and mixed micellar preparations that are useful within the disclosure include, but are not limited to, Na caprylate (C8), Na caprate (C10), Na laurate (C12) or Na oleate (C18), optionally combined with bile salts, such as glycocholate and taurocholate.

α-Interferons

The optional α-interferon component of the disclosure is preferably ALFERON N Injection® the only approved natural, multi-species, α-interferon available in the United States. It is the first natural source, multi-species interferon and is a consistent mixture of at least seven species of α-interferon. The interferon is preferably a natural cocktail of at least seven species of human α-interferon. In contrast, the other available α-interferons are single molecular species of α-interferon made in bacteria using DNA recombinant technology. These single molecular species of α-interferon also lack an important structural carbohydrate component because this glycosylation step is not performed during the bacterial process.

Unlike species of α-interferon produced by recombinant techniques, ALFERON N Injection® is produced by human white blood cells that are able to glycosylate the multiple α-interferon species. Reverse phase HPLC studies show that ALFERON N Injection® is a consistent mixture of at least seven species of alpha interferon (α2, α4, α7, α8, α10, α16 and α17). This natural-source interferon has unique antiviral properties distinguishing it from genetically engineered interferons. The high purity of ALFERON N Injection® and its advantage as a natural mixture of seven interferon species, some of which, like species 8b, have greater antiviral activities than other species, for example, species 2b, which is the only component of INTRON A®. The superior antiviral activities, for example, in the treatment of chronic hepatitis C virus (HCV) and HIV infection, and tolerability of ALFERON N Injection® compared to other available recombinant interferons, such as INTRON A® and ROFERON A®, have been reported. ALFERON N Injection® is available as an injectable solution containing 5,000,000 international units (IU) per ml.

For internal or any administration, the α-interferon may, for example, be formulated in conventional manner for oral, nasal or buccal administration. Formulations for oral administration include aqueous solutions, syrups, elixirs, powders, granules, tablets and capsules which typically contain conventional excipients such as binding agents, fillers, lubricants, disintegrants, wetting agents, suspending agents, emulsifying agents, preservatives, buffer salts, flavoring, coloring and/or sweetening agents. α-Interferon may be administered by any method of administration of this disclosure. Preferably administration is by a suitable route including oral, nasal, parenteral (including injection) or topical (including transdermal, buccal and sublingual). It will be appreciated that the preferred route will vary with the condition and age of the recipient, the nature of the infection and the chosen active ingredient.

The recommended dosage of the components will depend on the clinical status of the patient and the experience of the clinician in treating similar infection. As a general guideline, a dosage of ALFERON N Injection® utilized for systemic infections is 3 IU/pound to 10 million IU/pound (e.g., subcutaneous injection) three times weekly. Experience to date is with dosages above 3 IU/lb of patient body weight. Oral α-interferon (ALFERON LDO®) has been administered as a liquid solution in the range of 500-10,000 IU/day and calculated on the basis of a 150 pound human this is from 3.3 to 66.0 IU/lb per day. Our experience indicates beneficial results are obtained at dosage levels of α-interferon in excess of 450 IU, that is greater than 3 IU/pound body weight. A healthcare provider would be able, however, to determine the optimal dose and schedule of low dose oral α-interferon to achieve a desired antiviral effect.

Administration (Delivery)

In one aspect of the disclosure, Ebola transmission is blocked by administering to a subject to be exposed or exposed to Ebola by an amount of one or more dsRNAs effective to protect against viral infection or to mitigate the symptoms associated therewith. The administration of dsRNAs may be continued for at least from 24 hours to 72 hours, or until the subject's symptoms have improved.

In another aspect, a medicament (e.g., pharmaceutical composition) containing the immune activator(s) is provided. Optional other components of the medicament include excipients and a vehicle (e.g., aqueous buffer or water for injection) packaged aseptically in one or more separate containers (e.g., nasal applicator or injection vial). Processes for using and making the medicament are also provided. Further aspects will be apparent from the following description and claims, and any generalizations thereto.

The methods of the disclosure are useful for treating a subject in need thereof. A subject in need thereof is a subject having or at risk of having an Ebola virus infection. In its broadest sense, the terms “treatment” or “to treat” refer to both therapeutic and prophylactic treatments. If the subject in need of treatment is one who is at risk of having an Ebola virus infection, then treating the subject refers to reducing the risk of the subject having the infection or, in other words, decreasing the likelihood that the subject will develop Ebola Hemorrhagic Fever after exposure to Ebola virus, as well as to a treatment after the subject has been infected in order to fight the infectious disease, e.g., reduce or eliminate it altogether or prevent it from becoming worse.

Administration Format

The pharmaceutical composition comprising one or more active agents listed above may be administered to a subject by any local or systemic route known in the art including The pharmaceutical composition and/or the active agents may be micronized by milling or grinding solid material, dissolved in a vehicle (e.g., sterile buffered saline or water) for injection or instillation (e.g., spray), topically applied, or encapsulated in a liposome or other carrier for targeted delivery. It will be appreciated that the preferred route may vary with the age, condition, gender, or health status of the subject; the nature of disease or other pathological conditions, including the number and severity of symptoms; and the chosen active ingredient.

Administration of the compositions of the disclosure, including compositions comprising a vaccine, may be by any methods including, at least, intravenous administration; intradermal administration; subcutaneous administration; intramuscular administration; intranasal administration; intraperitoneal administration; intracranial administration; intravesical administration; oral administration (through the mouth, by breathing through the mouth); topical administration; inhalation administration; aerosol administration; intra-airway administration; tracheal administration; bronchial administration; instillation administration; bronchoscopic instillation administration; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; dry powder inhalants administration; and a combination thereof.

Some administration methods may be grouped differently or may be referred to by broader terms. For example, enteral administration may refer to oral administration, feeding tube administration, or enema administration; topical administration may be by a device such as a nebulizer for inhalation through the respiratory system, by skin patch acting epicutaneously or transdermally, or by suppository acting in the rectum or vagina. Parenteral administration may take the form of subcutaneous administration, intravenous administration, intramuscular administration, intradermal administration, or intraperitoneal injection or administration; buccal administration, sublingual administration, or transmucosal administration; inhalation administration, instillation administration, instillation administration intranasally or instillation administration intratracheally.

Nasal administration refers to any administration through the airway and is another term for pulmonary airway administration.

As a further example, in nasal administration or any administration, administration may include administering to a tissue selected from the group consisting of: an airway tissue; nose tissue; oral tissue; alveoli tissue; pharynx tissue; trachea tissue; bronchi tissue; carina tissue; bronchi tissue; bronchioles tissue; lung tissue; tissue in the lobe of a lung; alveoli tissue; nasal passage tissue; nasal epithelium tissue; larynx tissue; bronchi tissue; inhalation tissue; and a combination thereof.

In another example, any administration would include administration to at least to a cell selected from the group consisting of: an epithelium cell; an airway epithelium cell; a ciliated cell; a goblet cell; a non-ciliated cell; a basal cell; a lung cell; a nasal cell; a tracheal cell; a bronchial cell; a bronchiolar epithelial cell; an alveolar epithelial cell; a sinus cell; and a combination thereof.

Administration may be from a delivery system selected from the group consisting of: a nebulizer; a sprayer; a nasal pump; a squeeze bottle; a nasal spray; a syringe sprayer or plunger sprayer (a syringe providing pressure to an attached sprayer or nozzle); a nasal aerosol device; a controlled particle dispersion device; a nasal aerosol device; a nasal nebulization device; a pressure-driven jet nebulizer; ultrasonic nebulizer; a breath-powered nasal delivery device; a atomized nasal medication device; an inhaler; a powder dispenser; a dry powder generator; an aerosolizer; an intrapulmonary aerosolizer; a sub-miniature aerosolizer; a propellant based metered-dose inhalers; a dry powder inhalation devices; an instillation device; an intranasal instillation device; an intravesical instillation device; a swab; a pipette; a nasal irrigation device; a nasal rinse; an aerosol device; a metered aerosol device; a pressurized dosage device; a powdered aerosol; a spray aerosol; a spray device; a metered spray device; a suspension spray device; and a combination thereof.

Administration Formulation

Formulations for administration (i.e., pharmaceutical compositions) may include pharmaceutically acceptable carrier with the tdsRNA.

Pharmaceutical carriers include suitable non-toxic vehicles in which a composition of the disclosure is dissolved, dispersed, impregnated, or suspended, such as water or other solvents, fatty materials, celluloses and their derivatives, proteins and their derivatives, collagens, gelatine, polymers, adhesives, sponges, fabrics, and the like and excipients which are added to provide better solubility or dispersion of the drug in the vehicle. Such excipients may include non-toxic surfactants, solubilizers, emulsifiers, chelating agents, binding materials, lubricants softening agents, and the like. Pharmaceutically acceptable carriers may be, for example, aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents.

A liquid carrier may be present in the composition in a concentration effective to serve as a suitable vehicle for the compositions of the present disclosure. In general, the carrier is used in an amount of about 40 to about 98 wt. %, or about 50 to about 98 wt. % of the composition. The compositions of the present disclosure are preferably delivered as nasal sprays.

Unless otherwise indicated, all percentages (%) are meant to represent weight percent (wt %).

The liquid carrier may be water or any other suitable liquid, solvent, or mixture thereof. An antigen may be dispersed or dissolved in the liquid carrier in a therapeutically effective amount. The water may contain suitable buffering agents to result in a pH wherein the particular antigen is delivered optimally, or it may contain other carriers, such as glycerin, propylene glycol, polyethylene glycols of various sizes, amino acid modifiers, such as arginine and the like, and other suitable soluble excipients, as is known to those who are proficient in the art of compounding or pharmaceutics.

The preferred formulation may vary with the age, condition, gender, or health status of the subject, the nature of the disease or other pathological condition, including the number and severity of symptoms, and the chosen active ingredient.

The tdsRNA in solid form may be dissolved using known diluents for administration such as, for example, physiological phosphate-buffered saline, and then infused intravenously. The tdsRNA may be a combination or any subset of dsRNA described above. It is understood that in one aspect, tdsRNA may comprise a combination of all of the examples of tdsRNA described above or any subset of the above examples. With respect to the subsets, the specific exclusion of one or more specific embodiment of tdsRNA is also envisioned. As non-limiting examples, tdsRNA may comprise any of the following or any combination thereof: (1) any one of the examples of tdsRNA, (2) any combination of one or more of the examples of tdsRNA, (3) all of the examples of tdsRNA as described above, (4) any combination of one or more of the examples of tdsRNA and excluding any one or more examples of tdsRNA, (5) all of the examples of tdsRNA described above but without rIₙ•r(C₁₁₋₁₄U)ₙ, (6) Rugged dsRNA, (7) AMPLIGEN® (rIₙ•r(C₁₂U)ₙ) and Rugged dsRNA, (8) tdsRNA as described above but without rIₙ•r(C₁₁₋₁₄U)ₙ and without Rugged dsRNA.

The composition of the present disclosure may exist in various forms, for example, an oil-in-water emulsion, a water-in-oil emulsion, and a water-in-oil-in-water emulsion. The active compounds of the present disclosure, including the embodiments where tdsRNA is in combination with other agents, may exist in either the continuous or the dispersed phase or in both phases depending upon whether the compounds are hydrophilic, lipophilic, or amphiphilic. As an example, the emulsion comprises oil droplets dispersed in a continuous aqueous phase with a lipophilic enhancer being contained in the oil droplets and a water-soluble pharmaceutically active compound dissolved in the continuous aqueous phase. In a preferred embodiment wherein an oil phase is utilized, the concentration of the oil in the oil phase is such that it does not promote crystallization.

The composition of the present disclosure may also comprise an emulsifying agent for use in aiding the formation of an emulsion. Essentially any suitable hydrocolloid emulsifying agent, typically a solid material, or a mixture of two or more such emulsifying agents can be used in the practice of the present disclosure. Hydrocolloid emulsifying agents include: vegetable derivatives, for example, acacia, tragacanth, agar, pectin, and carrageenan; animal derivatives, for example, gelatin, lanolin, cholesterol, and lecithin; semi-synthetic agents, for example, methylcellulose and carboxymethylcellulose; and synthetic agents, for example, acrylic emulsifying agents such as carbomers. The hydrocolloid emulsifying agent forms hydrocolloids (hydrated lyophilic colloids) around the emulsified liquid droplets of the emulsion. The hydrocolloid serves as a protective layer around each emulsified droplet which physically repulses other droplets, thus hindering Ostwald ripening (the tendency of emulsified droplets to aggregate).

In contrast, other emulsifying agents typically protect the emulsified droplets by forming a liquid crystalline layer around the emulsified droplets. In compositions which employ a liquid crystalline layer-forming emulsifying agent, the hydrophilic-lipophilic balance (HLB) of the oil phase of the emulsion must be matched with that of the emulsifying agent to form a stable emulsion and, often, one or more additional emulsifying agents (secondary emulsifying agents) must be added to further stabilize the emulsion. The aforementioned liquid crystalline layer also retards the release of the compounds of the dispersed phase upon contact with the target substrate.

The hydrocolloid emulsifying agents for use in the composition of the present disclosure include compounds which exhibit a low level of irritability or no irritability to the target membrane and which have good bioadhesive and mucoadhesive properties. Examples of hydrocolloid emulsifying agents which exhibit such properties include cellulosic emulsifying agents and acrylic emulsifying agents, including, for example, those which have an alkyl group containing from about 10 to about 50 carbon atoms. Particularly preferred acrylic emulsifying agents for use in the present disclosure are copolymers of a carboxylic acid and an acrylic ester (described, for example, in U.S. Pat. No. 3,915,921 to Schlatzer and U.S. Pat. No. 4,509,949 to Huang et al.), with those which are cross-linked being especially preferred.

The emulsifying agent is present in the composition in a concentration that is effective to form the desired liquid emulsion. In general the emulsifying agent is used in an amount of about 0.001 to about 5 wt. % of the composition, and more generally in an amount of about 0.01 to about 5 wt. % of the composition, and most generally in an amount of about 0.1 to about 2 wt. % of the composition.

The composition of the present disclosure may include, as an optional ingredient, particulate solids dispersed in the composition. For example, the composition may include an additional pharmaceutically-active compound dispersed in the liquid continuous phase of the emulsion in the form of microcrystalline solids or nanoparticulates.

The liquid compositions are particularly suited for nasal administration.

Nasal Compositions

In one embodiment, a composition for enhancing intranasal delivery includes a combination of tdsRNA and active compounds (e.g., Ebola Vaccine) prepared for nasal delivery. The combination of tdsRNA and active compounds may be applied in a subsequent manner or a simultaneous manner. In a preferred embodiment, the mixture will be in the form of an aqueous solution. In other embodiments, the mixture will be a powder or a dried, powdered, or lyophilized form of the mixture. In some embodiments, these forms will be re-hydrated before delivery.

Each of the agents and chemicals described herein, including any combinations thereof, may be added to a tdsRNA for administration, including nasal administration, to a subject.

Medicament

In another aspect, a medicament (e.g., a pharmaceutical composition) containing the tdsRNA is provided. Optional other components of the medicament include excipients and a vehicle (e.g., aqueous buffer or water for injection) packaged aseptically in one or more separate containers (e.g., nasal applicator or injection vial). Further aspects will be apparent from the disclosure and claims herein.

Dosage for any Form of Administering

Dose Per Day for the Average Subject:

For a subject (e.g., 150 lb or 70 Kg human) the dose of dsRNA per day may be at least one selected from the group consisting of: 0.1 to 1,000,000 μg, 0.1 μg to 25,000 μg, 0.4 to 400,000 μg, 0.5 μg to 5,000 μg, 0.5 mg to 60 mg, 5 mg to 40 mg, 5 mg to 400 mg, 10 mg to 20 mg, 10 mg to 800 mg, 25 mg to 700 mg, 20 mg to 200 mg, 50 mg to 150 mg, 80 mg to 140 mg, and a combination thereof.

Dose in Kilogram Per Day:

In another aspect, the tdsRNA is administered in a dose per day selected from the group consisting of 0.1 mg/kg, 0.2 mg/kg, 0.3 mg/kg, 0.5 mg/kg, 0.7 mg/kg, 0.8 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 0.1-1 mg/kg, 0.1-2 mg/kg, 0.1-3 mg/kg, 0.1-4 mg/kg, 0.1-5 mg/kg, 0.1-6 mg/kg, 0.1-7 mg/kg, 0.1-8 mg/kg, 0.1-10 mg/kg, 0.1-20 mg/kg, 0.2-3 mg/kg, 0.3-3 mg/kg, 0.4-3 mg/kg, 0.6-3 mg/kg, and 0.8-3 mg/kg.

Amount Per Unit Dose:

The amount per unit dose of tdsRNA may be at least one selected from 0.1 mg/kg, 0.2 mg/kg, 0.4 mg/kg, 0.6 mg/kg, 0.8 mg/kg, 1 mg/kg, 2 mg/kg, 3 mg/kg, 4 mg/kg 5 mg/kg.

### Specific Examples

In one embodiment, the tdsRNA is administered at a dose from about 1 mg/kg to 10 mg/kg biweekly. As another example, the administration may be in 50-1400 milligrams every other day leading to an average daily dosage of 25-700 milligrams per day. In one embodiment, the tdsRNA is administered at a dose from about 0.50 mg/kg to 10 mg/kg every other week. 50-1400 milligrams every other day leading to an average daily dosage of 25-700 milligrams per day.

Dose Frequency:

In certain embodiments, the tdsRNA is administered at a frequency selected from the group consisting of: one dose per day, one dose every 2 days, one dose every 3 days, one dose every 4 days, one dose every 5 days, 4 doses a week, 3 doses a week, 2 doses a week, 1 dose a week, once every two weeks, once every three weeks, once every four weeks, and once a month.

Number of Doses and Dosing Period:

In certain embodiments, the tdsRNA is administered as a single dose, in two doses, in three doses, in four doses, in five doses, or in 6 or more doses. In other embodiments, the dosage is continued indefinitely. Continuous dosage may be used, for example, for a worker in a hospital constantly exposed to Ebola.

A dosing period is usually about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, and, in one embodiment, 6, 7, 8, 9, 10, 11, 12, 13, or 14 days, for example, 7 or 14 days. In certain embodiments, multiple (for example, 2, 3, 4, 5, 6, 7, 8, 9, 10 or more) doses of a tdsRNA are administered to a subject in need of treatment. As discussed, for a subject constantly exposed to Ebola such as a hospital or laboratory worker, the dosing period may be continuous without end.

Nasal Dosage:

tdsRNA may be administered at the same dose in nasal administration as for any other form of administration. Nonlimiting specific examples of nasal administration (which is also applicable for any other form of administration) include: a dose of 5 μg to 10 μg; 10 μg to 20 μg; 20 μg to 50 μg; 50 μg to 100 μg; 100 μg to 200 μg; 200 μg to 500 μg; 500 μg to 1000 μg; 1000 μg to 1500 μg; 1500 μg to 2000 μg; or any combination thereof.

Compositions and Methods that are Generally Applicable and Particularly Applicable for Nasal Administration

Compositions (Nasal Formulations) Preferred for Nasal Administration

Unless otherwise specified, “composition,” “a composition,” or “the composition” includes, at least, a composition of the disclosure or includes at least tdsRNA. Compositions may be optionally filtered and sterilized to enhance safety, stability and solubility.

In one embodiment, a composition for enhancing intranasal delivery includes tdsRNA and optionally active compounds prepared for nasal delivery. The combination of tdsRNA and active compounds may be applied in a subsequent (sequential) manner or a simultaneous (parallel) manner. In a preferred embodiment, the mixture will be in the form of an aqueous solution. In other embodiments, the mixture will be a powder or a dried, powdered, or lyophilized form of the mixture. In some embodiments, these forms will be re-hydrated before delivery. The composition may be in solid, liquid or any other form such as gels and liposomes.

A composition of the disclosure (e.g., tdsRNA) that is used in nasal administration is considered a nasal composition. Compositions of the disclosure are not limited to nasal administration. That is, any composition of the disclosure may be used as a nasal composition. Similarly, nasal compositions may be used for any other purposes such as non-nasal administration.

Simultaneous administration (also called parallel administration) may also comprise administration of two or more compositions at the same time. For example, two or more separate nasal nozzles and sprayers can each dispense a different composition for simultaneous administration. Simultaneous administration may also dispense compositions of different forms. For example, a dry powder and a liquid may be dispensed together in separate sprayers at the same time.

Each of the agents and chemicals described herein, including any combinations thereof, may be administered together with a composition of the disclosure (e.g., tdsRNA), nasally or otherwise, to a subject. Non-limiting examples of other compounds for nasal administration include RNA, DNA, adjuvants, proteins, interferons, Ebola virus (intact, inactivated, attenuated) or parts thereof. Non-limiting examples of these parts would include, at least, unpurified, semi-purified and purified parts. Ebola virus, and especially parts thereof, may be collected from at least one selected from the group consisting of an Ebola virus, an Ebola virus culture grown in a laboratory (in vitro), Ebola virus collected from an animal, Ebola virus collected from the wild (e.g., from a diseased animal), a cloned or and genetically engineered Ebola virus, an in vitro synthesized Ebola virus or parts thereof (e.g., cell free in vitro synthese), a synthetic Ebola antigen (e.g., from a peptide synthesizer), Ebola virus expressed from a transgenic organism (e.g., transgenic mammal, yeast, bacteria or the like).

As discussed, the Ebola virus includes “parts thereof.” Non-limiting examples of these parts include at least one selected from the group consisting of protein including recombinant protein, nucleic acid including DNA, RNA, synthetic nucleic acid, and combinations thereof (e.g., combinations of synthetic and natural nucleic acid in a double strand), antigens, peptides.

Preferred embodiments of compounds for administration include tdsRNA, Ebola virus or parts thereof including inactivated or attenuated forms and antigens thereof.

We note that tdsRNA is stable as a solid or dissolved in water and therefore any additional component is optional. Other components may benefit from additional ingredients described herein.

In certain embodiments, the therapeutic agent is administered with an agent that disrupts, e.g., transiently disrupts, tight junctions, such as EGTA (see U.S. Pat. No. 6,855,549).

Furthermore, since nasal administration may be perceived by a sense of smell in the subject, additives that improve the fragrances or nasal acceptance or reduce irritation may be added. These include buffers and preservatives if the composition is not made sterile, for example, methyl hydroxybenzoate, antioxidants, flavoring agents, volatile oils, buffering agents and surfactants.

### Specific Examples of Compositions

Aerosol compositions can be made with liquid and dried compositions of the disclosure to be administered via inhalation. These aerosol compositions can be placed into pressurized acceptable propellants, such as dichlorodifluoromethane, propane, and nitrogen. Compositions may be formulated as pharmaceuticals for non-pressured preparations, such as in a nebulizer or an atomizer. For compositions to be administered from multiple dose containers, antimicrobial agents can be added.

Liquid solutions may be suitable for any administration including nasal administration. Liquid compositions may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. The composition of the disclosure can be administered in a physiologically acceptable diluent in a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

The compositions may be formulated as dry, semidry, or liquid particles. The particulate pharmaceutical composition may optionally be combined with a carrier to aid in dispersion or transport. A suitable carrier such as a sugar (i.e., dextrose, lactose, sucrose, trehalose, mannitol) may be blended with the active compound or compounds in any suitable ratio.

Specific examples of compositions forms include at least the following: aerosol of liquid, aerosol suspension of respirable solid, dry powder inhalants, metered-dose inhalants, liquid/liquid suspensions, emulsions, suspensions, oil in water emulsion, and water in oil emulsions.

In reference to particles or droplets, it is envisioned that a particle or a droplet may be a solid, a liquid, or other types of particle such as a gel, a liposome, and the like. Also, it is envisioned that a composition may be dispensed as one type of particle but is delivered to a subject as a second type of particle. For example, a composition may be dispensed as a liquid particle with a high evaporation rate such that the liquid is transformed into a solid by the time the particle reaches the subject.

Certain devices require the use of various compositions suitable for the dispensing of some compositions of the present disclosure. Typically, each composition is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified systems may also be prepared in different compositions depending on the type of chemical modification or the type of device employed.

Compositions suitable for use with a nebulizer may also include a buffer and a simple sugar (e.g., for stabilization of the composition and regulation of osmotic pressure). The carrier is typically water (and most preferably sterile, pyrogen-free water) or a dilute aqueous alcoholic solution, preferably made isotonic, but may be hypertonic with body fluids by the addition of, for example, sodium chloride. The nebulizer composition may also contain a surfactant to reduce or prevent surface induced aggregation caused by atomization of the solution in forming the aerosol. Optional additives include preservatives if the composition is not made sterile, for example, methyl hydroxybenzoate, antioxidants, flavoring agents, volatile oils, buffering agents and surfactants.

Compositions for use with a metered-dose inhaler device may generally comprise a finely divided powder (a composition of the disclosure) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Compositions for dispensing from a powder inhaler device may comprise a finely divided dry powder containing a composition as described herein, and may also include a bulking agent, such as lactose, sorbitol, sucrose, or mannitol in amounts which facilitate dispersal of the powder from the device, e.g., 50 to 90% by weight of the composition. The composition may be prepared in particulate form with an average particle size of less than 10 mm (or microns), most preferably 0.5 to 5 mm, for most effective delivery to the distal lung.

Non-limiting specific examples of nasal (pulmonary) administration include at least one or more of the administration methods such as: oral administration (through the mouth, by breathing through the mouth); intranasal administration (e.g., by nose drops); inhalation administration; aerosol administration; intra-airway (e.g., tracheal or bronchial) administration; bronchoscopic instillation; intratracheal administration; mucosal administration; dry powder administration; respiratory administration; instillation administration.

Another example of nasal administration includes any deposition to any part of the airway, including, for example, by spray, by a swab, intratracheal deposition, intrabronchial deposition and bronchoscopic deposition, nasal rinse, nasal lavage, a temporary or permanent depot implant.

Administration by “inhalation” may be performed using a composition of the disclosure of a size sufficiently small to pass through the mouth or nose and larynx, past the oropharyngeal region, upon inhalation and into the bronchi and alveoli of the lungs. In general, particles (droplets, liquid or solid) ranging from about 1 to 10 microns in size (more particularly, less than about 5 microns in size) are respirable and suitable for administration by inhalation. The particles can be solid or liquid. In some embodiments, such preparations have a mean particle size of 4, 5, 6, 7, 8, 9, 10, 11, 12, or 13 microns.

In some embodiments, preparations for inhaled or aerosol delivery are formulated as a dry powder. In some embodiments, preparations for inhaled or aerosol delivery are formulated as a wet powder, for example through inclusion of a wetting agent. in some embodiments, the wetting agent is selected from the group consisting of water, saline, or other liquid of physiological pH. In some embodiment, the particles may be a liquid.

Administration by intranasal administration may be performed by particles of a larger size formulated and delivered to treat topically the nasal epithelium. Particles or droplets used for intranasal administration generally have a diameter that is larger than those used for administration by inhalation. For intranasal administration, a particle size in the range of 10-500 microns is preferred to ensure retention in the nasal cavity.

In some embodiments, particles for inhalation and particles for intranasal administration may be administered together. That is, particles of 1 to 500 microns are used. In some embodiments, particles of 1-10 or 1-13 microns are selected for or enriched. In other embodiments, particles of 10-500 microns, or 15 to 500 micron are selected for or enriched.

The compositions of the disclosure may be administered as a plurality of drops to the nasal or buccal cavity. A dose may be, for example, 1-100, 1-50, 1-20, 1-10, 1-5, drops.

In some embodiments, inventive compositions are administered using a device that delivers a metered dosage of composition.

Aerosols of liquid particles of the compositions of the disclosure may be produced by any suitable means, such as with a nebulizer, pressure-driven jet nebulizer, an ultrasonic nebulizer, or other means.

Aerosols of solid particles comprising the composition of the disclosure may likewise be produced with any solid particulate therapeutic aerosol generator. One illustrative type of solid particulate aerosol generator is an insufflator. Suitable compositions for administration by insufflation include finely comminuted powders which may be delivered by means of an insufflator or taken into the nasal cavity in the manner of a snuff. In the insufflator, the powder (e.g., a metered-dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the composition of the disclosure or of a powder blend comprising the composition and a suitable powder diluent, such as lactose, and an optional surfactant. The composition of the disclosure typically comprises from 0.1% to 100% w/w of the composition.

Another type of illustrative aerosol generator comprises a metered-dose inhaler. Metered-dose inhalers are pressurized aerosol dispensers, typically containing a suspension or solution composition of the tdsRNA in a liquefied propellant. During use these devices discharge the composition through a valve adapted to deliver a metered volume, typically from 10 μl to 200 μl, to produce a fine particle spray containing the tdsRNA. Suitable propellants include certain chlorofluorocarbon compounds, for example, dichlorodifluoromethane, trichlorofluoromethane, dichlorotetrafluoroethane and mixtures thereof. The composition may additionally contain one or more co-solvents, for example, ethanol, surfactants, such as oleic acid or sorbitan trioleate, antioxidant and suitable flavoring agents.

The preferred route and mode of administration will vary with the condition and age of the recipient, the nature of the infection or condition, and the chosen active ingredient.

Nasal Administration Devices

A device, encompassing a composition of the disclosure is also an embodiment.

The composition of the disclosure may be delivered by any nasal administration device or combination of devices. A combination refers to a composition that is both administered by two different devices or a device having the feature of two devices. Non-limiting examples of suitable devices that can be use individually or together include at least one selected from the group consisting of: a nebulizer; a sprayer (e.g., a spray bottle such as “Nasal Spray Pump w/Safety Clip, Pfeiffer SAP #60548; a squeeze bottle (e.g., bottle commonly used for nasal sprays, including ASTELIN (azelastine hydrochloride, Medpointe Healthcare Inc.) and PATANASE (olopatadine hydrochloride, Alcon, Inc.); a nasal pump spray (e.g., APTAR PHARMA nasal spray pump); a controlled particle dispersion devices (e.g., VIANASE electronic atomizer); a nasal aerosol device (e.g., ZETONNA nasal aerosol); a nasal nebulization device (e.g., EASYNOSE nebulizer, a pressure-driven jet nebulizer, or an ultrasonic nebulizer); a powder nasal delivery devices (e.g., OPTINOSE breath-powered nasal delivery device); an atomized nasal medication device (e.g., LMA MAD NASAL device); an instillation device; an inhalation device (e.g., an inhaler); a powder dispenser; a dry powder generator; an aerolizer (e.g., intrapulmonary aerosolizer or a sub-miniature aerosolizer, metered aerosol, powdered aerosol, spray aerosol); a spray; a metered spray; a metered dose inhalers (e.g., a propellant based metered-dose inhaler); a dry powder inhalation device; an intranasal instillation device; an intravesical instillation device; an insufflation device.

An application device for application to mucous membranes, such as, that of the nose, throat, and/or bronchial tubes (i.e., inhalation). This can be a swab, a pipette or a device for nasal irrigation, nasal rinse, or nasal lavage.

Another example is a syringe or plunger activated sprayer. This could be, for example, a sprayer head (or nozzle) attached, for example, via a Luer lock, to a syringe. The syringe applies a pressure to a composition that flows through the sprayer head and produces a spray or an aerosol.

Exemplary Kits

The disclosure also includes kits. The kit has a container housing an inhibitor of the disclosure (e.g., dsRNAs, interferons) and optionally additional containers with other therapeutics such as anti-Ebola agents or Ebola vaccines. The kit also includes instructions for administering the component(s) to a subject who has or is at risk of having an Ebola virus infection.

In some aspects of the disclosure, the kit can include a pharmaceutical preparation vial, a pharmaceutical preparation diluent vial, and an inhibitor. The vial containing the diluent for the pharmaceutical preparation is optional. The diluent vial contains a diluent such as physiological saline for diluting what could be a concentrated solution or lyophilized powder of inhibitor. The instructions can include instructions for mixing a particular amount of the diluent with a particular amount of the concentrated pharmaceutical preparation, whereby a final formulation for injection or infusion is prepared.

The instructions may include instructions for use in an oral formulation, inhaler, intranasal sprayer, intravenous injection or any other device useful according to the disclosure. The instructions can include instructions for treating a patient with an effective amount of inhibitor. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

### Discussion of Further Embodiments and Features

Subject or Patient

As used herein, a “subject” has the same meaning as a “patient” and is a mammal, preferably, a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. Other examples of subjects include swine, cattle, horses, camels, cats, dogs, rodents, birds, bats, rabbits, ferrets, mink, and the like. As used herein, the terms “patient” or “subject” are used interchangeably.

Devices and Kits

In another aspect, the present disclosure relates to and comprises a therapeutic device for intranasal delivery. In one embodiment, the therapeutic device may comprise any suitable devices charged with a preparation of tdsRNA and optionally, another biologically active agent such as a vaccine or antigen. These devices are described in more detail below.

Additional Methods and Compositions

In any aspect of this disclosure, the method may comprise a further step of administering to the subject one or more compound or agent selected from the group consisting of: antiviral, interferon, interferon mixture, Alferon, alpha-interferon species, recombinant or natural interferon-alpha, recombinant or natural interferon-alpha-2a, recombinant or natural interferon-beta, recombinant or natural interferon-beta-1b, and recombinant or natural interferon-gamma.

The alpha-interferon species may be a mixture of at least seven species of alpha-interferon produced by human white blood cells. The seven species may be, for example, interferon alpha 2, interferon alpha 4, interferon alpha 7, interferon alpha 8, interferon alpha 10, interferon alpha 16, and interferon alpha 17.

In another aspect, the agent may be one or more selected from the group consisting of Remdesivir, chloroquine, hydroxychloroquine, oseltamivir, zanamivir, abacavir, zidovudine, zalcitabine, didanosine, stavudine, efavirenz, indinavir, ritonavir, nelfinavir, amprenavir, ribavirin, interleukin, IL-2, PD-L1, Anti-PD-L1, checkpoint inhibitor, peramivir, and neuraminidase inhibitors.

The compositions and methods of this disclosure may comprise any compound/agent discussed herein including, e.g., in this previous paragraph.

Effective Amount: Therapeutically or Prophylactically Effective Amount

The compositions are delivered in effective amounts. The term “effective amount” refers to the amount necessary or sufficient to realize a desired biologic effect. Combined with the teachings provided herein, by choosing among the various active compounds and weighing factors such as potency, relative bioavailability, patient body weight, severity of adverse side effects and preferred mode of administration, an effective prophylactic or therapeutic treatment regimen can be planned which does not cause substantial toxicity and yet is effective to treat the particular subject to effectively preventing, treating, inhibiting, or attenuating an Ebola virus infection.

In addition, based on testing, toxicity of the inhibitor is expected to be low. The effective amount for any particular application can vary depending on such factors as the disease or condition being treated, the particular inhibitor being administered, the size of the subject, or the severity of the disease or condition. One of ordinary skill in the art can empirically determine the effective amount of a particular active ingredient without necessitating undue experimentation. It is preferred generally that a maximum dose be used, that is, the highest safe dose according to some medical judgment. Multiple doses per day may be contemplated to achieve maximum level of protection against Ebola virus.

For any compound described herein, the therapeutically effective amount can be initially determined from preliminary in vitro studies and/or animal models. A therapeutically effective dose can also be determined from human data for inhibitors that have been tested in humans and for compounds that are known to exhibit similar pharmacological activities, such as other related active agents. The applied dose can be adjusted based on the relative bioavailability and potency of the administered compound. Adjusting the dose to achieve maximal efficacy based on the methods described above and other methods well known in the art, is well within the capabilities of the ordinarily skilled artisan.

Ebola Virus Vaccine

One embodiment of the disclosure relates to tdsRNA used alone.

Another embodiment of the disclosure relates to tdsRNA administered with an Ebola vaccine. An Ebola vaccine comprises one or more antigens that can trigger an immune response and produce immunity to Ebola in a host subject. The compositions of this disclosure may contain one or more Ebola antigens and the composition of this disclosure can be used for immunization against Ebola.

Ebola has been grown in culture (e.g., Vero E6 cell cultures) and Ebola antigens have been identified and expressed (e.g., Ebola proteins GP, nucleoprotein, VP24, VP30, VP35 and VP40).

Methods of inactivating a virus and using the virus as a component of a vaccine are known. The United States Department of Agriculture has approved protocols for using binary ethylene-imine or formaldehyde to inactivate certain viruses for vaccine production. These methods are disclosed in numerous publications such as, for example, in U.S. Pat. Nos. 5,459,073; 5,811,099; 5,849,517; 5,811,099; 5,849,517; 7,252,984; 8,278,083 and published U.S. Patent Appl. 2011/0110975. These patents and patent applications are incorporated herein by reference.

Vaccines and antigens that may be used in the present compositions, for example, in combination with tdsRNA, include, but are not limited to, Ebola proteins GP, nucleoprotein, VP24, VP30, VP35 and VP40, and peptides from such proteins preferably of 6 amino acids in length or longer. Alternatively, antigen may be a protein fragment that is genetically engineered or the results of a protease digestion. Antigens can also be killed, attenuated or inactivated virus as well as semi purified fractions thereof. An antigen may be a nucleic acid, including DNA and RNA, that encodes an antigen and which can cause expression of the antigen when administered to a subject (host) causing, for example, expression of the antigen or a part thereof.

The compositions of this disclosure may contain a vaccine that has one type of antigen or more than one type of antigen. The antigen is present in the composition in a therapeutically effective amount. In general the antigen is present in an amount of about 0.001 to about 50 wt. % of the composition, about 0.01 to about 30 wt. %, about 0.1 to about 20 wt. %, about 0.1 to about 10 wt. %, or about 0.1 to about 2 wt. % of the composition.

The antigen of the present disclosure may be used in a comparatively crude state, or may be purified before use. For purification, for example, a method conventionally used in the art for the purification of a peptide, protein, DNA, RNA, carbohydrate, may be carried out in the present disclosure, such as filtration, concentration, centrifugation, gel filtration chromatography, ion exchange chromatography, hydrophobic chromatography, adsorption chromatography, high performance liquid chromatography, affinity chromatography, gel electrophoresis, isoelectric focusing and the like. When necessary, these methods may be combined as appropriate. According to the form of final use, purified antigen may be concentrated or freeze-dried to give a liquid or solid.

At least one immunological adjuvant may be used in the present composition to assist or modify the action of an antigen. Immunological adjuvants may lead to one or more of the following effects, among others: an increased immune response, a more diversified immune response, an accelerated immune response, a more persistent/prolonged immune response. Adjuvants that may be used in the present disclosure include, but are not limited to, dextran or cyclodextran and saponin.

Non-limiting examples of adjuvants include: (1) aluminum salts (alum), such as aluminum hydroxide, aluminum phosphate, aluminum sulfate, etc.; (2) submicron emulsions comprising a metabolizable oil, such as squalene, and an emulsifying agent, such as one or more sorbitan derivatives; (3) MF59 containing 5% squalene, 0.5% Tween 80, and 0.5% Span 85 (optionally containing various amounts of MTP-PE (see below), although not required) formulated into submicron particles; (4) SAF, containing 10% squalane, 0.4% Tween 80, 5% pluronic-blocked polymer L121, and thr-MDP (see below) either microfluidized into a submicron emulsion or vortexed to generate a larger particle size emulsion; (5) Ribi adjuvant system (RAS), (Ribi Immunochem, Hamilton, Mont.); (6) saponin adjuvants, such as Quil A, or QS21; (7) Complete Freunds Adjuvant (CFA) and Incomplete Freunds Adjuvant (IFA); (8) cytokines; (9) phospholipid adjuvants, including lipopolysaccharide and liposaccharide phosphate adjuvants; (10) a polyoxyethylene ether or a polyoxyethylene ester. For additional examples of immunological adjuvants, see Vaccine Design, The Subunit and the Adjuvant Approach, Powell, M. F. and Newman, M. J, eds., Plenum Press, 1995.

Carrier and Additional Components

By way of illustration, the inactivated Ebola virus may be mixed with a suitable carrier (e.g., water or saline) that optionally is buffered (e.g., phosphate buffered saline, such as Dulbecco's phosphate buffered saline “D-PBS”) before administering into a subject animal as a vaccine. Preferably, the carrier is such that the inactivated virus is uniformly dispersed in the resulting composition at the time of the administration, and it will not degrade the antigen-treated virus throughout a storage life of at least 10 days, more preferably at least one month at a temperature of about 0° C. to about 37° C. An example of one suitable solution includes a mixture of CaCl₂); MgCl₂; KCl; KH₂PO₄; NaCl; Na₂HPO₄; and D-Glucose (dextrose). More specifically, one example of such a solution is CaCl₂) at 0.901 mM; MgCl₂ at 0.493 mM; KCl at 2.67 mM; KH₂PO₄ at 1.47 mM; NaCl at 137.93 mM; Na₂HPO₄ at 8.06 mM; and D-Glucose (dextrose) at 5.56 mM.

A carrier or diluent for the vaccine may include one or any combination of stabilizers, preservatives and buffers. Suitable stabilizers may include, for example, SPGA, carbohydrates (such as sorbitol, mannitol, starch, sucrose, peptone, arginine, dextran, glutamate or glucose), proteins (such as dried milk serum, albumin or casein) or degradation products thereof. Suitable buffers may include for example alkali metal phosphates. Suitable preservatives may include thimerosal, merthuilate and gentamicin. Diluents include water, aqueous buffer (such as buffered saline) and polyols (such as glycerol). It will be appreciated that vaccine compositions herein, as well as any of its carrier or diluents are preferably free of any antibiotic, and/or any mercury-containing ingredient.

The vaccine may further comprise an adjuvant or additional reagent, such as an adjuvant selected from one or any combination of lecithin, a pharmaceutically acceptable polymer, saponin or a derivative thereof, or cholesterol. One preferred adjuvant or additional reagent is tdsRNA.

Optionally, a unit dosage of inactivated Ebola virus or virus antigen may be as follows. For example, a dosage may be, for example, about 1 μg, about 5 μg, about 10 μg, about 20 μg, about 25 μg, about 30 μg, about 50 μg, about 100 μg, about 125 μg, about 150 μg, or about 200 μg. Alternatively, a dosage is less than about 1 μg, (for example about 0.08 μg, about 0.04 μg; about 0.2 μg, about 0.4 μg, about 0.8 μg, about 0.5 μg or less, about 0.25 μg or less, or about 0.1 μg or less), or more than about 125 μg, (for example about 150 μg or more, about 250 μg or more, or about 500 μg or more).

The dosages of (1) tdsRNA and (2) Ebola virus antigen (or inactivated Ebola virus) are disclosed and where a composition or method or mixture comprising both are made the dosage of each can be used for the combination.

The composition, including compositions comprising vaccines containing antigens of the disclosure, may be used to protect or treat an animal, such as a mammal, susceptible to Ebola virus infection, by means of administering said vaccine via systemic or more specific routes. Any administration method of this disclosure may be used for the composition and vaccine. Specific examples of preferred embodiments are discussed below. Nasal vaccination methods are not particularly limited as long as it can induce an immune response, for example, an immune response in the topical mucous membrane of the respiratory tract (particularly upper respiratory tract), which is an infection route of many immunogen such as bacterium and virus. Any methods of nasal administration of this disclosure may be used. As another example, administration may include injection via the intramuscular, intraperitoneal, intradermal or subcutaneous routes; or via mucosal administration to the oral/alimentary, respiratory, genitourinary tracts.

Other Aspects

General Discussion

In this specification, stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity that a person skilled in the art would understand does not affect the operation of the disclosure or its patentability.

All modifications and substitutions that come within the meaning of the claims and the range of their legal equivalents are to be embraced within their scope. A claim which recites “comprising” allows the inclusion of other elements to be within the scope of the claim, the disclosure is also described by such claims reciting the transitional phrases “consisting essentially of” (i.e., allowing the inclusion of other elements to be within the scope of the claim if they do not materially affect operation of the disclosure) or “consisting of” (i.e., allowing only the elements listed in the claim other than impurities or inconsequential activities which are ordinarily associated with the disclosure) instead of the “comprising” term. Any of these three transitions can be used to claim the disclosure.

It should be understood that an element described in this specification should not be construed as a limitation of the claimed disclosure unless it is explicitly recited in the claims. Thus, the granted claims are the basis for determining the scope of legal protection instead of a limitation from the specification which is read into the claims. In contradistinction, the prior art is explicitly excluded from the disclosure to the extent of specific embodiments that would anticipate the claimed disclosure or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the disclosure. Similarly, generalizations of the disclosure's description are considered to be part of the disclosure.

From the foregoing, it would be apparent to a person of skill in this art that the disclosure can be embodied in other specific forms without departing from its spirit or essential characteristics. While the disclosure has been described in connection with what is presently considered to be the most practical and preferred embodiment, it is to be understood that the disclosure is not to be limited to the disclosed embodiment, but on the contrary, is intended to cover various modifications and equivalent arrangements included within the spirit and scope of the appended claims.

### INCORPORATION BY REFERENCE

All publications, patent applications, and patents mentioned herein are hereby incorporated by reference in their entirety as if each individual publication or patent was specifically and individually indicated to be incorporated by reference. These patents include, at least, U.S. Pat. Nos. 4,024,222, 4,130,641, 5,258,369, 7,439,349, 8,722,874 and 9,315,538. In case of conflict, the present application, including any definitions herein, will control.

### EXAMPLES

### Example 1 Evaluation of dsRNA (Including AMPLIGEN®) for Prevention of Ebola Virus Transmission

We assessed the ability of tdsRNA, specifically AMPLIGEN®, to produce resistance to virus transmission. The evaluation was performed with intraperitoneal (i.p.) administration of AMPLIGEN®. The virus was an Ebola virus (EBOV variant guinea pig-adapted Mayinga: GP-EVOV) from infected guinea pigs. The guinea pig has been a commonly used model for investigating the efficacy of drugs inhibiting Ebola transmission for more than 20 years. See, e.g., https://www.the-scientist.com/?articles.view/articleNo/41837/title/Guinea—Pigs—to—Model—Ebola—Spread/. See also, Ryabchilkova et al., Ebola virus infection in guinea pigs: presumable role of granulomatous inflammation in pathogenesis, Arch Virol. 1996; 141(5): 909-21; Marzi, Evaluation of Ebola Virus Countermeasures in Guinea Pigs, Methods Mol Biol. 2017; 1628:283-291.

There are three groups of animals. (1) “Transmitter animals” were infected directly with Ebola. (2) “Treated animals” were treated with tdsRNA (AMPLIGEN® in this case). Treating involved administering 10 mg/kg intraperitoneal doses of tdsRNA (AMPLIGEN®) to animals at minus 24 hours (i.e., 24 hours before zero hour), 48 hours and 96 hours. (3) “Untreated animals” were a control group. The untreated animals were kept under the same conditions as the treated animals, except they did not receive tdsRNA but received PBS instead.

The treated animals received tdsRNA (AMPLIGEN®) 24 hours before zero hour, and 48 hours and 96 hours after zero hour. Zero hour is defined as the initiation of exposure between infected and uninfected-treated animals. Exposure was confirmed because every exposed animal that was tested was seropositive for anti-Ebola antibodies.

The transmitter guinea pigs received a lethal dose of GP-EBOV given intranasally (i.n.). The intranasal route of infection causes lethal pneumonia in guinea pigs and ensures that the virus will be readily transmitted to contact animals. Control transmitter guinea pigs were given PBS.

In the experiment, pre-infection, and pre-treatment pre-study weights were taken for all animals, and a baseline serum was collected (saphenous vein).

At −24 hours, twelve transmitter guinea pigs were infected with a 10,000×LD50 (220 PFU) of GP-EBOV by the intranasal route 24 hours before zero hour. Six “treated animals” were treated with 10 mg/kg tdsRNA (AMPLIGEN®) given by the intraperitoneal route 24 hours before zero hour.

At zero hour, the 12 infected animals were weighed and oral and rectal swabs and nasal washes were collected.

At zero hour, the GP-EBOV infected animals (transmitter animals) were housed with the uninfected animals in the same cage but separated by a barrier to prevent physical contact. That is, while the air is shared and some bedding may be shared, there is no physical contact between the infected transmitter animals and the “treated animals” or the “untreated animals.” Six tdsRNA-treated animals were housed together with 6 infected animals (transmitter animals) in a single cage. Similarly, six PBS control animals (untreated animals) were are housed with 6 infected animals (transmitter animals) in a single cage.

Equal numbers of male and female animals were used in the study. The intended design is that 6 animals were housed in one caging unit (ferret cage unit of dimensions 2×3 ft) in groups.
6a(i). Group 1—3 male-infected+3 male PBS-treated contacts
6a(ii). Group 2—3 female-infected+3 female PBS-treated contacts
6a(iii). Group 3—3 male-infected+3 male AMPLIGEN®-treated contacts
6a(iv). Group 4—3 female-infected+3 female AMPLIGEN®-treated contacts

All animals were visually assessed daily for clinical signs of illness.

Swabs and nasal washes were collected and animals were weighed according to the following schedule (with day 1=day that infected and contact animals are housed together in the same cage):

Transmitter animals—days 1, 3, 5, 7, 9, 11, 13 (animals will typically die by day 10).

Contact animals (“treated animals” and “untreated animals”—days 2, 4, 6, 8, 10, 12, 14.

Results:

All the transmitter animals that were infected with 10,000 LD50 of the GP-EBOV died between days 7 and 9 post-infection. All the untreated animals—the animals treated with PBS and not treated with tdsRNA—died at about the same time frame. These results demonstrate Ebola virus infection in all animals and a uniformly lethal outcome.

Of the five animals that received tdsRNA (AMPLIGEN®) and were infected with Ebola, 3 animals survived indicating a survival rate of 60% for tdsRNA treated animals vs. 0% for PBS treated animals for animals that were exposed to Ebola while being housed with infected animals. All the surviving animals showed seroconversion—indicating positive exposure to Ebola.

To determine the long term durability of the protective effects of tdsRNA, the surviving animals were exposed to a lethal dose (10,000×LD50 dose) of Ebola at 42 days (42 days since day zero). Briefly, all the “treated animals” were infected with 10,000×LD50 (220PFU) of Ebola virus intranasally. These animals were monitored daily and weighed and scored for clinical signs of illness. Of the animals tested, 66% survived being challenged by this high dose of Ebola virus. These results show that tdsRNA stimulates strong resistance in the treated animals even 42 days after administration. The survival of the 10,000×LD50 challenge is remarkable since such a high dosage does not occur regularly in nature. It is also surprising because the last tdsRNA dose was administered on day 14 and, therefore, the 10,000×LD50 challenge was performed 28 days after the last administration of tdsRNA.

The swabs and blood samples that were collected during this study at scheduled time points remain archived in Biosafety Level 4 (−80° C.) storage. In our study, tdsRNA (AMPLIGEN®) provides a positive outcome in 60% (3/5) of the animals that were infected. Further, in addition to surviving exposure to Ebola at zero hour, the animals showed durable resistance to unnaturally high levels of Ebola—up to 66% of the animals survived an Ebola exposure directly applied and at a dosage that is 10,000 times higher than the dose that would kill 50% of exposed animals. As our controls have shown, no animal untreated with tdsRNA survived such a high titer challenge.

### Example 2 Evaluation of dsRNA (Including AMPLIGEN®) for Early Treatment of Ebola Virus Transmission in a Second Animal Model

Similar to Example 1, we assessed the ability of tdsRNA, specifically AMPLIGEN®, to produce resistance to virus transmission in a second animal model—the mouse and specifically the BALB/c mouse. The evaluation was performed with intraperitoneal (i.p.) administration of AMPLIGEN®. The virus was mouse adapted Ebola virus. In this experiment, we tested to see if tdsRNA can provide resistance and treatment after exposure to Ebola.

Ten animals were used per group. The groups were treated as follows:

10 mice were treated with PBS (i.e., 0 mg/kg tdsRNA); 10 mice were treated with 6 mg/kg tdsRNA; 10 mice were treated with 12 mg/kg tdsRNA; 10 mice were treated with 18 mg/kg tdsRNA. In each case, treatment involved 7 doses. One dose each was given at day 0, day 2, day 4, day 6, day 8, day 10, and day 12.

The animals were first infected once at 1000 pfu with Ebola. The first tdsRNA was administered 4 hours after the infection and the mice were observed for 21 days post infection. As discussed above, further tdsRNA was administered at day 2, day 4, day 6, day 8, day 10, and day 12.

Results:

After 7 days, all control animals (0 mg/kg tdsRNA) died. In contrast, 100% of the 6 mg/kg tdsRNA survived. One animal in the 18 mg/kg tdsRNA group died on day 8 and one animal in the 12 mg/kg tdsRNA died on day 9. Other than those two deaths, all the animals treated with 12 mg/kg tdsRNA and 18 mg/kg tdsRNA survived.

The results clearly indicate that tdsRNA, administered even 4 hours after exposure to Ebola, can increase survival to 90% or 100% depending on dosage.

Discussion:

Without wishing to be bound by theory or mechanism of action, the generation of protective immunity may depend not only on exposure to antigen but also on the context in which the antigen is encountered. Numerous examples exist in which the introduction of a novel antigen into a host generates tolerance, or no reaction, rather than long-term immunity. The presentation of an antigen, such as those of Ebola, in the presence of tdsRNA may be able to induce long-term immunity. The tdsRNA does not have to be present simultaneously with Ebola, but exposure to tdsRNA within a sufficient time before or after exposure to Ebola can (1) stimulate an innate resistance to Ebola and (2) allow a higher therapeutic/toxicity ratio for Ebola antigen for developing a protective long-term immunity. A higher therapeutic/toxicity ratio means that a lower dose of Ebola can be sufficient to induce an effective long-term immunity in a host. Since Ebola infection is often lethal, a higher therapeutic/toxicity ratio is obviously desirable.

Our results show that exposure to Ebola by itself, without tdsRNA stimulation, can result in tolerance or an inadequate immune response. This results in the death of the subject. However, prompt treatment with tdsRNA (in this case AMPLIGEN®), even after exposure to Ebola virus, can prevent the occurrence of symptoms, or at least prevent the occurrence of serious symptoms including death. In this example and in the previous example, it is clear that at the very least, tdsRNA slows down, inhibits, or attenuates Ebola replication. Further, tdsRNA clearly can prevent and treat Ebola virus infections. In the case of a lethal pathogen such as Ebola, a proper immune response be developed because tdsRNA has prevented, treated, inhibited, or attenuated the Ebola virus's replication. This can mean the difference between ineffective immunity (including tolerance), or effective immunity; or life or death in a subject that is exposed to, or is about to be exposed to Ebola virus.

1. A method of preventing, treating, inhibiting, or
attenuating an Ebola virus infection of a subject, the method comprising the step of:
administering an effective amount of a composition comprising a tdsRNA; and
a pharmaceutically acceptable carrier; to the subject
thereby preventing, treating, inhibiting, or attenuating the Ebola virus infection of the subject.

2. The method of claim 1 wherein the administering is performed within a period of time of from 96 hours before to 96 hours after exposure to Ebola virus; from 72 hours before to 72 hours after exposure to Ebola virus; from 48 hours before to 48 hours after exposure to Ebola virus; from 24 hours before to 24 hours after exposure to Ebola virus; from 12 hours before to 12 hours after exposure to Ebola virus; from 6 hours before to 6 hours after exposure to Ebola virus; from 3 hours before to 3 hours after exposure to Ebola virus; from 1 hour before to 1 hour after exposure to Ebola virus; within 4 days after exposure to Ebola virus; within 3 days after exposure to Ebola virus; within 2 days after exposure to Ebola virus; within 1 day after exposure to Ebola virus; within 12 hours after exposure to Ebola virus; within 6 hours after exposure to Ebola virus; within 3 hours after exposure to Ebola virus; and within 1 hour after exposure to Ebola virus.

3. The method of claim 1 wherein the method attenuates the replication of Ebola virus in the subject.

4.-5. (canceled)

6. A composition for preventing, treating, inhibiting, or attenuating an Ebola virus infection of a subject comprising a tdsRNA, and a pharmaceutically acceptable carrier.

7. The method of claim 1, wherein the composition does not further comprise an active ingredient; does not further comprise an active ingredient that is an antigen; does not contain an antigen from the Ebola virus; does not contain a nucleic acid with a nucleic acid sequence that is at least 90% identical to an Ebola virus nucleic acid; or does not contain a wildtype Ebola virus nucleic acid.

8. The method of claim 1, wherein the composition further comprises one or more selected from the group consisting of: an absorption-promoting agent; a delivery-enhancing agent; a mucolytic agent; a mucus clearing agent; a ciliostatic agent; a penetration-promoting agent; a permeation-promoting agent; a vasodilator agent; a vasoconstrictor agent; RNase inhibitory agent; an enzyme inhibitor; a selective transport-enhancing agent; a stabilizing delivery vehicle; a carrier; a support; and a complex-forming species.

9. The method of claim 1, wherein the subject is converted from seronegative for Ebola to seropositive for Ebola after exposure to Ebola virus without symptoms of Ebola virus infection.

10. The method of claim 1, wherein the method produces immune resistance to Ebola virus infection in the subject after exposure to Ebola virus.

11. The method of claim 10, wherein the immune resistance to Ebola virus infection persists for at least 10 days, at least 20 days, at least 30 days, at least 40 days, at least 50 days, at least 2 months, at least 3 months, at least 4 months, at least 6 months, at least 1 year, or at least 2 years.

12. The method of claim 1, wherein the composition further comprises a natural mixture of human alpha interferons.

13. The method of claim 1, wherein the subject is a mammal, a human, or a nonhuman animal.

14. The method of claim 1, wherein the tdsRNA is selected from the group consisting of rIₙ•r(C₄₋₂₉U)ₙ; rIₙ•r(C₁₁₋₁₄U)ₙ; rIₙ•r(C₄U)ₙ; rIₙ•r(C₅U)ₙ; rIₙ•r(C₆U)ₙ; rIₙ•r(C₇U)ₙ; rIₙ•r(C₈U)ₙ; rIₙ•r(C₉U)ₙ; rIₙ•r(C₁₀U)ₙ; rIₙ•r(C U)ₙ; rIₙ•r(C₁₂U)ₙ; rIₙ•r(C₁₃U)ₙ; rIₙ•r(C₁₄U)ₙ; rIₙ•r(C₁₅U)ₙ; rIₙ•r(C₁₆U)ₙ; rIₙ•r(C₁₇U)ₙ; rIₙ•r(C₁₈U)ₙ; rIₙ•r(C₁₉U)ₙ; rIₙ•r(C₂₀U)ₙ; rIₙ•r(C₂₁U)ₙ; rIₙ•r(C₂₂U)ₙ; rIₙ•r(C₂₃U)ₙ; rIₙ•r(C₂₄U)ₙ; rIₙ•r(C₂₅U)ₙ; rIₙ•r(C₂₆U)ₙ; rIₙ•r(C₂₇U)ₙ; rIₙ•r(C₂₈U)ₙ; rIₙ•r(C₂₉U)ₙ; rIₙ•r(C₃₀U)ₙ; rIₙ•r(C₃₁U)ₙ; rIₙ•r(C₃₂U)ₙ; rIₙ•r(C₃₃U)ₙ; rIₙ•r(C₃₄U)ₙ; rIₙ•r(C₃₅U)ₙ; rIₙ•r(C₄₋₃₀U)ₙ; rIₙ•r(C₁₄₋₃₀U)ₙ; rIₙ•r(C₁₁₋₁₄G)ₙ; rIₙ•r(C₄₋₂₉G)ₙ; rIₙ•r(C₃₀₋₃₅U)ₙ; r(Poly I•Poly C)ₙ; r(Poly A•Poly U)ₙ; and a combination thereof.

15. The method of claim 1, wherein the tdsRNA is resistant to denaturation under conditions that are able to separate hybridized poly(riboinosinic acid) and poly(ribocytosinic acid) strands (rIₙ•rCₙ).

16. The method of claim 14, wherein n is selected from the group consisting of: 40 to 50,000; 50 to 10,000; 60 to 9000; 70 to 8000; 80 to 7000; 40 to 500; 380 to 450; and a combination thereof.

17. The method of claim 1, wherein the tdsRNA comprises 1 mol % to 4 mol % rugged dsRNA or 4 mol % to 16 mol % rugged dsRNA.

18. The method of claim 1, wherein the tdsRNA comprises rIₙ•r(C₁₁₋₁₄U)ₙ; and rugged dsRNA.

19. The method of claim 18, wherein the rugged dsRNA has one or more properties selected from the group consisting of: 40-500 bp in length; 380-450 bp in length; 250 kDa to 320 kDa in molecular weight; 30-38 dsRNA helical turns in length; formula of rIₙ•r(C₄₋₂₉U)ₙ; formula of rIₙ•r(C₁₁₋₁₄U)ₙ; formula of rIₙ•r(C₁₂U)ₙ; formula of rIₙ•r(C₃₀U)ₙ; and formula of rIₙ•r(C₃₀₋₃₅U)ₙ.

20. The method of claim 1, wherein the tdsRNA has one or more physical properties selected from the group consisting of: about 4 to about 5000 helical turns of duplexed RNA; 30-38 helical turns of duplexed RNA; about 2 kilodaltons to about 30,000 kilodaltons molecular weight; and
about 250 kilodaltons to about 320 kilodaltons molecular weight.

21. The method of claim 1, wherein at least 30 weight percent of total tdsRNA in the composition is a linear structure; at least 40 weight percent of total tdsRNA in the composition is a linear structure; at least 50 weight percent of total tdsRNA in the composition is a linear structure; at least 60 weight percent of total tdsRNA in the composition is a linear structure; at least 70 weight percent of total tdsRNA in the composition is a linear structure; at least 80 weight percent of total tdsRNA in the composition is a linear structure; or at least 90 weight percent of total tdsRNA in the composition is a linear structure.

22. The method of claim 1, wherein the tdsRNA is complexed with a stabilizing polymer.

23. The method of claim 4-22, wherein the stabilizing polymer is one or more selected from the group consisting of polylysine; polylysine plus carboxymethylcellulose; polyarginine; polyarginine plus carboxymethylcellulose; carboxymethylcellulose; and a combination thereof.

24. The method of claim 1, wherein the composition is administered at a dosage of about 25-700 milligram of tdsRNA.

25. The method of claim 1, wherein the composition is administered at a rate which is at least one selected from the group consisting of: one dose per day; one dose every 2 days; one dose every 3 days; one dose every 4 days; one dose every 5 days; once a week; twice a week; 3 times a week; once every two weeks; once every 3 weeks; once every 4 weeks; and once a month.

26. The method of claim 12, wherein the natural mixture of human alpha interferons is a purified mixture of at least three different human interferon-alpha proteins with native amino acid sequences and glycosylation patterns.

27. The method of claim 12, wherein the natural mixture of human alpha interferons is administered in a dosage from 5 IU per pound body weight/day to 100,000 IU per pound body weight/day.

28. The method of claim 1, wherein administering is selected from the group consisting of: systemic administration; intravenous administration; intradermal administration; subcutaneous administration; intramuscular administration; nasal administration; intranasal administration; pulmonary airway administration; intraperitoneal administration; intracranial administration; intravesical administration; oral administration; intravaginal administration; intrarectal administration; intratracheal administration; oropharyngeal administration; sublingual administration; topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; dry powder inhalants administration; and a combination thereof.

29. The method of claim 1, wherein administering is by a delivery system selected from the group consisting of: a nebulizer; a sprayer; a nasal pump; a squeeze bottle; a nasal spray; a syringe sprayer; a plunger sprayer; a nasal aerosol device; a controlled particle dispersion device; a nasal aerosol device; a nasal nebulization device; a pressure-driven jet nebulizer; ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered-dose inhalers; a dry powder inhalation devices; an instillation device; an intranasal instillation device; an intravesical instillation device; a swab; a pipette; a nasal irrigation device; a nasal rinse; an aerosol device; a metered aerosol device; a pressurized dosage device; a powdered aerosol; a spray aerosol; a spray device; a metered spray device; a suspension spray device; and combination thereof.

30. The method of claim 1, wherein the composition is a prophylactic or therapeutic vaccine, wherein the vaccine comprises one or more Ebola antigens, an inactivated Ebola virus, or an attenuated Ebola virus.

31. The method of claim 30, wherein the composition is a nasal vaccine.

32. The method of claim 30, wherein the one or more Ebola virus antigens comprises an antigen purified from an Ebola virus or an inactivated Ebola virus.

33. The method of claim 30, wherein a combination of the tdsRNA and the Ebola antigen provides a vaccine effect that is superior than that of the Ebola antigen administered alone.
